SEQUENCE LISTING (16 sequences)

SEQ ID NO: 1 - protein, 700 aa, Pyrococcus sp. 12/1
  Pyrococcus sp. 12-1 DNA primase
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDKGDFRVLAERLVQELAPWFRRVKEKGGSRHHLVNAIAAYIARNTNLTFEDLAKIWEIVHAELVRLGLEDPNDWSNRYHTIKDVYEKLQAGTFLGTRAYMLKYLNMMSEEEIVEILRAVKRALFPYLKPVNAGFVSKFVDEPYGRDEAPKKWEDVDEDRRKAVGVFFIRALAFETAEYIYVEDLPKPKTFSIKSHSEDGKEKLRFNEALWQSFLSWLGVQEGEPFDRVDFENLLIEKFGITEHELRSIYFSKVLYLLTPEGMRMPKCIVEFLKELADEGNLSDEKVAHLAHWIKYFAKPLRHSTTSIMLRAQGKPVDMRMAVWAKIVEFFAEDDDVAKRLVSVFKKAYEEAEPPFPCFGVKECPFFPGHKGCPFIAPKRNEILAVSLVDVQIHGTDGIVVVVGGGE

SEQ ID NO: 2 - protein, 296 aa, Artificial Sequence
  PolpP12?297-898
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDK

SEQ ID NO: 3 - protein, 290 aa, Artificial Sequence
  PolpP12?87-92?297-898
MRPSDIIIDVYKAIQDHPGAGKLAIELRFYPRPTSEWIIVADIEDKAEELHKVLFKNNVLGKKEAYISMALHDFEEVGKKLEKLREQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDK

SEQ ID NO: 4 - protein, 928 aa, Thermococcus sp. CIR10
  Thermococcus sp. CIR10 DNA primase
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKKENFEELAKRLVEELAPWFKKIKERGGSRHHLVNAIAAYIARNTNLTEEDLLGKDQEDGTHVVGLWELVHSKLVELGLEDPGDWSNRYHTIKDVYEKLYAGTTLGTRAYMMKYLNVSEEEAIEILRSVKRALFPYLHPVNVQVISKFEAKPYSKEEAPTEWEAVDEDRKKAVGIWYIEVLALETANYVYIEDLSKPGVFYIVEKVKRTVKVGKKEKGVEVDEYHFNPALWQSFLNWLGIKEGEPIEREELWNLLLEKFDIKDYELRAIYFRKILHLLSPEGMRRPRCVEEFLRELADEGFLSEDKVRHLAHWIKFYAKPLRHSTTSIMLRAKGKPVDMRMAVWAKVVEFFAEDEETAQGLIETFREAYEQAEPPFPCFGARECPFFQEHRGCPFIAPKRDEILAVSLVDVQLHGSDGIVIIVGSEEGTKKFVHKGKVEWQKQGKSKIKYPVAEWFLDVYAKEFLSLPEAPSWSHEEVTEILKSRARVVRSQLNEFDEYFDNFIDWLRSENARGIYPYEKADSSHIFIKGNMIGIPPRLAEDFYRNELGISGRKFKEMLIRELGSYYLGKKAAWIKLSSGQHNGVNCYFISLDWFKKIVGEPNIKDIEAEGDIGSGGFNYEEEEGEA

SEQ ID NO: 5 - protein, 303 aa, Artificial Sequence
  PolpTCIR10?303-928
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKK

SEQ ID NO: 6 - protein, 914 aa, Thermococcus peptonophilus
  Thermococcus peptonophilus DNA primase
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKKEELETLALNLADELIPWFKKVKERGGSWHHLVNAIGAYVVRNTNLSLEDLIGKDNPDGTHVVGLWEIVFQRLVEKSAEDPGDWVNRRNTIKDVYEKHIAGKPLGTRAYLKKYLPVSDEEVVEILMAVRRALLPFLKEVKKVDSSGFGVAPYKKTAPRSWDEVDEDRKRATGRWYVWKLAFNTAEYLFTDELPKAGTFYIDVWMKEGKREWMKRFFNEALFRSFIEDGLGYKYGAPVEREELFERLVEVFNITDEEVRGIYIDAALSLLSPVGMRTPPCIEEFIMEFAANGSLSEDKVRHLARWIKLYAKPLKHSTTTTKLVGAGYKVDMRMAVWAKLVEFFAEDDEVARELVRVFKEEYGAAEPPFTCIGTKTCQFYLNEKMCPFIIPKEKEILAVSLIDVQRHESDGLVVIVGGDKEVRTFVKKGNVEWVKKTERREKYPVAEWFIDVFATEYLSVSPDDLDVDLEEVTDILKSRARVVKSRLNELEDMYEKFVEWLKRENAVRGVLPYEKADFNHLFIKGNMIGIPPALAEEFYRFELNIKGSEFREMLEKKLGFHYTKKAVKLSVGEKKDVRRCYLVSLEWFRKVVGEPNVKDVVMAGDIALSGLVYYESGEEVVE

SEQ ID NO: 7 - protein, 295 aa, Artificial Sequence
  PolpTpep?295-914
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKK

SEQ ID NO: 8 - protein, 913 aa, Thermococcus celericrescens
  Thermococcus celericrescens DNA primase
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKKERLETLALNLADELAPWFKKVKERGGSWHHLVNAIGAYIVRNTNLSLEDLIGKDNPDGTHVVGLWELVFQRLVEKGAEDPSDWLNRRNTIKDVYEKHIAGKPLGTRAYLKKYLPVSDEEVVEILMAARRALLPFLKGVKRAGSSGFGVFPYKDTAPRSWNEVEAERKRATGLWYVWSLAFSTAEYIFTDELSKAGTFFILVKEGKSIKSVFNETLFRSFIEEGLGYKYGMPVRRDELFERLVEVFNITDEEVRGVYIDAALSLLSPVGMRTPPCIEEFIMEFAANGNLPEDKVRHLARWIKLYAKPLKHSTTTTKLVGAGYNVDMRMAVWAKLVEFFAEDDEVAGELVRIFKEEYKEAEPPFTCIGARTCPFYLKDDVAKMCPFIFPKEKEILAVSLIDVQRHESDGIVILVGGAREVRTFIKKGKVEWVKRTKKTEKYPIAEWFIDVFATEYLGVPPDNLDFDLKDVTGILKSRERVVPSQLNEVEDWYEKFVEWLKRENEVRGVLPYKKADVHHLFIKDNMIGIPPALAEEFYRYEANIKPSEFREMLEVKLSIHYKKKAVKLSVGEKKDVRRCYLVSLEWFRKVVGEPNVKDVVMAGDIALSGLVYYESGEEVVE

SEQ ID NO: 9 - protein, 295 aa, Artificial Sequence
  PolpTcele?295-913
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKK

SEQ ID NO: 10 - protein, 923 aa, Thermococcus nautili
  Thermococcus nautili sp. 30-1 DNA primase
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTSSNFEALAERLFTELRPWWEIAKEKGWSRHHLTMGIATYILRNTNLTPEQLIGSENSPGLWELVFAKLVEAGLEDPDDWKNRASTIRDAYKKIESGKKVATKAYLRKYIEGLSEEDAVQILLSVKRALLPYLKAVDVKRISKYSARPYEITEEIPKSWEDIDENRKKATGVWYIDFLALETANDIYFEDLPKPPVFYIRFVEKNKEKFKLNETLYHSFLTWLGITEGEPLDRTELIDLLVEKFGFTVEDLKAIYYRKILTLLKPEGMRTPKCIQEFLFELATEGNLPEDKIRHLAHWVKFYARPLRHSTTSVLLKGRGKPVDMRLAIWAKVVEFFAEDDEVAEELITTFKKAYMGAEPPFPCIGAESCPFYPDHRACPFIVPKRKEVLPVSIVDVQLHGSDGIVVLVGGPTEVTAFTLEGKVEWIKTTKKTVKYPIAEWFLDRFAKEYLSLPEAPSWWKLEEVTEILKSRARVVKSQFDKFEDYLEQFIEWLQKENSRRGILPYEKADENHLFIKGEWVGIPPGFAREFYSGELLIGGPTFRRMLEQKLGKDYRKMSAKIHLNTGLKDKRNCYFVSVEWFRKHVGEPNIQEITSEGDVSFDGLSYDDEEEGVVG

SEQ ID NO: 11 - protein, 310 aa, Artificial Sequence
  PolpTN2?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 12 - protein, 347 aa, Pyrococcus furiosus
  DNA primase catalytic subunit PriS
MLMREVTKEERSEFYSKEWSAKKIPKFIVDTLESREFGFDHNGEGPSDRKNQYSDIRDLEDYIRATSPYAVYSSVAFYENPREMEGWRGAELVFDIDAKDLPLKRCNHEPGTVCPICLEDAKELAKDTLIILREELGFENIHVVYSGRGYHIRILDEWALQLDSKSRERILAFISASEIENVEEFRRFLLEKRGWFVLKHGYPRVFRLRLGYFILRVNVPHLLSIGIRRNIAKKILDHKEEIYEGFVRKAILASFPEGVGIESMAKLFALSTRFSKAYFDGRVTVDIKRILRLPSTLHSKVGLIATYVGTKEREVMKFNPFRHAVPKFRKKEVREAYKLWRESLEYE

SEQ ID NO: 13 - protein, 346 aa, Thermococcus kodakarensis
  DNA primase catalytic subunit PriS
MSKLLREVTPEERRLYYSGEWDAKKLPEFIVESIERREFGFDHTGEGPSDRKNAFSDVRDLEDYIRATAPYAAYSSVAFYRNPQEMEGWLGAELVFDIDAKDLPLRRCQNEHPSGQVCPICLEDAKELARDTLIILKEDFGFENIHVVYSGRGYHIRVIDEWALKLDSKARERILSYVSAAEEVTFDDIQKRYIMLSSGYFRVFRLRFGYFIQRINENHLKNIGLKRSTAEKLLDEKTRQDIVEKFVNKGLLAAFPEGVGYRTLLRLFGLSTTFSKAYFDGRVTVDLKRILRLPSTLHSKVGLVATYIGSDEKRLEKFDPFKDAVPEFRKEEVQKAYQEWKELHEG

SEQ ID NO: 14 - protein, 330 aa, Sulfolobus solfataricus
  DNA primase small subunit PriS
MGTFTLHQGQTNLIKSFFRNYYLNAELELPKDMELREFALQPFGSDTYVRHLSFSSSEELRDYLVNRNLPLHLFYSSARYQLPSARNMEEKAWMGSDLLFDIDADHLCKLRSIRFCPVCGNAVVSEKCERDNVETLEYVEMTSECIKRGLEQTRNLVEILEDDFGLKPKVYFSGNRGFHVQVDCYGNCALLDSDERKEIAEYVMGIGVPGYPGGSENAPGWVGRKNRGINGVTIDEQVTIDVKRLIRIPNSLHGKSGLIVKRVPNLDDFEFNETLSPFTGYTIFLPYITIETEVLGSIIKLNRGIPIKIKSSIGIYLHLRNLGEVKAYVR

SEQ ID NO: 15 - protein, 346 aa, Pyrococcus horikoshii [sequence omitted]

SEQ ID NO: 16 - protein, 335 aa, Archaeoglobus fulgidus
  DNA primase small subunit PriS
MLTKLFLKKKFEEYYSKNEVELPRKFKNREFAFVPLELLPDFVMHRHISFRSETDFRAYILSNVPAHIYFSSAYYERPAEDKMENKGWLGADLIFDIDADHLPVKAQSFEKALEMAKREIKKLTAVLRADFGIRDMKIYFSGGRGYHVHVHDEEFLSLGSAERREIVDYLRLNSPKIVVEDRFANSNAAKRVLNYLRKKLEEDERLTSKLKIKPADLKKEKLTKKVIRAVEKFDYSALSIYIDAPVTADVKRLIRLPGSLHGKTGLRVTEVEDIESFNPLKDALAFGDEAVVVKVARKLNLSIGDFSGKIYPGRVKLPEYAAVFLICRGDASYDS

(19) United States
(12) Patent Application Publication
(10) Pub. No.: US 2023/0235373 A1
(43) Pub. Date: Jul. 27, 2023

(54) CONTROLLED TEMPLATE-INDEPENDENT SYNTHESIS OF NUCLEIC ACIDS USING THERMOSTABLE ENZYMES

(71) Applicant: SYNHELIX, Evry-Courcouronnes (FR)

(72) Inventors: Irina RANDRIANJATOVO-GBALOU, Evry-Courcouronnes (FR); Ahmed SAID, Evry-Courcouronnes (FR); Renaud RAHIER, Evry-Courcouronnes (FR)

(21) Appl. No.: 18/009,673

(22) PCT Filed: Jun. 11, 2021
(86) PCT No.: PCT/EP2021/065867

§ 371 Date: Dec. 9, 2022

Related U.S. Application Data
(60) Provisional application No. 63/038,168, filed on Jun. 12, 2020.

(30) Foreign Application Priority Data
Aug. 6, 2020 (EP) 20305903.5

(51) Int. Cl. C12P 19/34; C12N 9/12

(52) U.S. Cl. CPC C12P 19/34; C12N 9/1247; C12Y 207/07006

(57) ABSTRACT

The invention relates to methods for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of an archaeal DNA primase or a functionally active fragment and/or variant thereof, thereby covalently binding said nucleoside triphosphate to the free 3-hydroxyl group of the 3-end nucleotide. It also relates to isolated functionally active fragments of archaeal DNA primases which are capable of template-independent terminal nucleotidyl transferase activity but are devoid of a template-independent primase activity.

### FIELD OF THE INVENTION

The invention relates to the field of nucleic acid synthesis or sequencing, more specifically to methods for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of an archaeal DNA primase or a functionally active fragment and/or variant thereof, thereby covalently binding said nucleoside triphosphate to the free 3′-hydroxyl group of the 3′-end nucleotide.

It also relates to isolated functionally active fragments of archaeal DNA primases which are capable of template-independent terminal nucleotidyl transferase activity but are devoid of ab-initio single-stranded nucleic acid synthesis activity.

### BACKGROUND OF THE INVENTION

Template-independent, sequence-controlled synthesis of nucleic acids represents a major industrial challenge.

Many industries are capable of synthesizing DNA or RNA strands by chemical means; however, they have quickly experienced the limits of these manufacturing processes. Today, the gold method for chemical synthesis of nucleic acids is the phosphoramidite method, developed in the 1980's, and later enhanced with solid-phase supports and automation (Beaucage & Caruthers, 1981. Tetrahedron Lett. 22(20):1859-62; McBride & Caruthers, 1983. Tetrahedron Lett. 24(3):245-8; Beaucage & Iyer, 1992. Tetrahedron. 48(12):2223-2311).

However, this method shows major limitations: first, nucleic acids with no more than around 250 nucleotides can be synthetized. Second, the phosphoramidite method requires the use of organic solvents which can be carcinogens, reproductive hazards, and neurotoxins; while synthetic byproducts can further be toxic and polluting.

In order to overcome these problems, a new method of template-independent, sequence-controlled nucleic acid synthesis by enzymatic means has recently been developed. It is based on the use of a terminal deoxynucleotidyl transferase (TdT), an enzyme which is able to polymerize single-stranded DNA fragments in the absence of template strand. This “template-independent” property was hence exploited for the sequence-controlled synthesis of nucleic acids, using reversibly 3′-blocked nucleoside triphosphates.

However, the use of TdT also has its own limits, in particular during the polymerization of long nucleic acids, or of sequences rich in guanine-cytosine. Indeed, in these two cases, the synthetized DNA strand tends to fold in on itself and to form secondary structures, thereby masking the 3′ position of the strand and ultimately leading to a drastic reduction in the final synthesis yield.

Methods are being explored to work around this problem. In particular, authors in Singapore have recently developed an assay to identify thermostable TdT variants (Chua et al., 2020. ACS Synth Biol. 9(7):1725-1735). In brief, they generated a library of TdT mutants using an error-prone polymerase, and screened about 10000 of these TdT mutants. They finally identified one TdT variant that was 10° C. more thermostable than the wildtype TdT of bovine origin (which optimum temperature is around 37° C., with an unfolding Tₘ around 40° C.), while preserving its catalytic properties. In the same time, another research group has reported a similar outcome using an in silico-driven approach to identify a TdT variant that was 10° C. more thermostable than the wildtype TdT from Mus musculus (Barthel et al., 2020. Genes (Basel). 11(1):102).

Although promising, this finding does not yet resolve all the issues explained above, and their remains a need for an enzyme which is capable of template-independent, sequence-controlled synthesis, at temperatures between 60° C. and 95° C., to avoid the formation of any secondary structures and increase the final nucleic acid synthesis yield.

The present invention offers such means and methods.

### SUMMARY

The present invention relates to a method for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of a primase domain of an archaeal DNA primase belonging to the primase-polymerase family or a functionally active variant thereof capable of template-independent terminal nucleotidyl transferase activity but devoid of ab-initio single-stranded nucleic acid synthesis activity, thereby covalently binding said nucleoside triphosphate to the free 3′-hydroxyl group of the 3′-end nucleotide.

In one embodiment, said archaeal DNA primase or the functionally active variant thereof is from an archaeon of the Pyrococcus genus. In one embodiment, said archaeal DNA primase or the functionally active variant thereof is Pyrococcus sp. 12-1 DNA primase. In one embodiment, said archaeal DNA primase belonging to the primase-polymerase family or the functionally active variant thereof is Pyrococcus sp. 12-1 DNA primase having the amino acid sequence of SEQ ID NO: 1.

In one embodiment, said primase domain of an archaeal DNA primase belonging to the primase-polymerase family is the primase domain of the Pyrococcus sp. 12-1 DNA primase having the amino acid sequence of SEQ ID NO: 2 or SEQ ID NO: 3, or a functionally active fragment and/or variant thereof:
having at least 70% sequence identity with the amino acid sequence of SEQ ID NO: 2 or SEQ ID NO: 3; and
being capable of template-independent terminal nucleotidyl transferase activity; and
being devoid of an ab-initio single-stranded nucleic acid synthesis activity.

In one embodiment, said primase domain of an archaeal DNA primase belonging to the primase-polymerase family is the primase domain of the Pyrococcus sp. 12-1 DNA primase having the amino acid sequence of SEQ ID NO: 2, or a functionally active fragment and/or variant thereof:
having at least 70% sequence identity with the amino acid sequence of SEQ ID NO: 2; and
being capable of template-independent terminal nucleotidyl transferase activity; and
being devoid of an ab-initio single-stranded nucleic acid synthesis activity.

In one embodiment, the initiator sequence is immobilized onto a support. In one embodiment, the initiator sequence is a single stranded nucleic acid primer.

In one embodiment, the template-independent synthesis of nucleic acids is carried out at a temperature ranging from about 60° C. to about 95° C.

In one embodiment, said method is for template-independent synthesis of nucleic acids with random nucleotide sequence, and the at least one nucleoside triphosphate, or the combination of nucleoside triphosphates, does not comprise terminating nucleoside triphosphates.

In one embodiment, said method is for template-independent sequence-controlled synthesis of nucleic acids, and the at least one nucleoside triphosphate is a terminating nucleoside triphosphate comprising a reversible 3′-blocking group.

In one embodiment, the method comprises the steps of:
a) providing the initiator sequence comprising a 3′-end nucleotide with a free 3′-hydroxyl group;
b) contacting said 3′-end nucleotide with a reversibly terminating nucleoside triphosphate in the presence of the primase domain of the archaeal DNA primase belonging to the primase-polymerase family or the functionally active variant thereof,

thereby covalently binding said reversibly terminating nucleoside triphosphate to the free 3′-hydroxyl group of the 3′-end nucleotide;
c) applying a washing solution to remove all reagents, in particular to remove unbound reversibly terminating nucleoside triphosphates;
d) cleaving the reversible 3′-blocking group of the covalently bound terminating nucleoside triphosphate in the presence of a cleaving agent;

thereby obtaining a nucleotide with a free 3′-hydroxyl group;
e) optionally, applying a washing solution to remove all reagents, in particular to remove the cleaving agent;
f) optionally, reiterating steps b) to e) multiple times to synthetize the nucleic acid until desired length and nucleotide sequence.

The present invention also relates to an isolated functionally active fragment of an archaeal DNA primase consisting of an amino acid sequence of SEQ ID NO: 2 or SEQ ID NO: 3, or a functionally active fragment and/or variant thereof:
having at least 70% sequence identity with the amino acid sequence of SEQ ID NO: 2 or SEQ ID NO: 3; and
being capable of template-independent terminal nucleotidyl transferase activity; and
being devoid of an ab-initio single-stranded nucleic acid synthesis activity.

In one embodiment, the isolated functionally active fragment of the archaeal DNA primase or variant thereof consists of an amino acid sequence of SEQ ID NO: 2, or a functionally active fragment and/or variant thereof:
having at least 70% sequence identity with the amino acid sequence of SEQ ID NO: 2; and
being capable of template-independent terminal nucleotidyl transferase activity; and
being devoid of an ab-initio single-stranded nucleic acid synthesis activity.

In one embodiment, the isolated functionally active fragment of the archaeal DNA primase or variant thereof consists of the amino acid sequence of SEQ ID NO: 2 or SEQ ID NO: 3.

The present invention also relates to a nucleic acid encoding the functionally active fragment of an archaeal DNA primase according to the invention.

The present invention also relates to an expression vector comprising the nucleic acid according to the invention, operably linked to regulatory elements, preferably to a promoter.

The present invention also relates to a host cell comprising the expression vector according to the invention.

The present invention also relates to a method of producing the functionally active fragment of an archaeal DNA primase according to the invention, said method comprising:
(a) culturing the host cell according to the invention, under conditions suitable for the expression of said functionally active fragment of the archaeal DNA primase or variant thereof; and
(b) isolating said functionally active fragment of the archaeal DNA primase or variant thereof from said host cell.

The present invention also relates to a kit comprising:
an initiator sequence comprising a 3′-end nucleotide with a free 3′-hydroxyl group, optionally immobilized onto a support;
at least one nucleoside triphosphate, optionally wherein the at least one nucleoside triphosphate is a terminating nucleoside triphosphate comprising a reversible 3′-blocking group; and
the isolated functionally active fragment of the archaeal DNA primase according to the invention.

### DETAILED DESCRIPTION

In a first aspect, the present invention relates to an isolated functionally active fragment of an archaeal DNA primase or variant thereof; a nucleic acid encoding the same; an expression vector comprising the latter; a host cell comprising this expression vector; and a method of production of said isolated functionally active fragment of an archaeal DNA primase or variant thereof.

“DNA primase” refer to enzymes involved in the replication of DNA, belonging to the class of RNA polymerases. They catalyze de novo synthesis of short RNA molecules called primers, typically from 4 to 15 nucleotides in length, from ribonucleoside triphosphates in the presence of a single stranded DNA template. DNA primase activity is required at the replication fork to initiate DNA synthesis by DNA polymerases (Frick & Richardson, 2001. Annu Rev Biochem. 70:39-80).

“Isolated” and any declensions thereof, as well as “purified” and any declensions thereof, are used interchangeably when with reference to an archaeal DNA primase or a functionally active fragment thereof, and mean that said archaeal DNA primase or functionally active fragment thereof is substantially free of other components (i.e., of contaminants) found in the natural environment in which said archaeal DNA primase or functionally active fragment thereof is normally found. Preferably, an isolated or purified archaeal DNA primase or functionally active fragment thereof is substantially free of other proteins or nucleic acids with which it is associated in a cell. By “substantially free”, it is meant that said isolated or purified archaeal DNA primase or functionally active fragment thereof represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Functionally active fragment”, with reference to an archaeal DNA primase, means a fragment or a domain of an archaeal DNA primase which is capable of template-independent terminal nucleotidyl transferase activity, while being, preferably, devoid of an ab-initio single-stranded nucleic acid synthesis activity. Means and methods to assess the activity of a fragment or a domain of an archaeal DNA primase are well known to the one skilled in the art. These include the assays described in the Example section of the present disclosure, and others, such as those described by Guilliam & Doherty (2017. Methods Enzymol. 591:327-353).

In one embodiment, the isolated functionally active fragment of the archaeal DNA primase or variant thereof according to the present invention is capable of template-independent terminal nucleotidyl transferase activity. In one embodiment, the isolated functionally active fragment of the archaeal DNA primase or variant thereof according to the present invention is devoid of an ab-initio single-stranded nucleic acid synthesis activity. In one embodiment, the isolated functionally active fragment of the archaeal DNA primase or variant thereof according to the present invention is capable of template-independent terminal nucleotidyl transferase activity but devoid of an ab-initio single-stranded nucleic acid synthesis activity.

By “template-independent terminal nucleotidyl transferase activity”, it is meant the addition of nucleoside triphosphates to the 3′ terminus of a nucleic acid molecule, in absence of complementary nucleic acid template.

By “ab-initio single-stranded nucleic acid synthesis activity” or “template-independent primase activity”, it is meant the synthesis of single stranded nucleic acid molecules in absence of both complementary nucleic acid template and initiator sequence, i.e., starting from a single nucleotide.

In one embodiment, the archaeal DNA primase belongs to the archaeo-eukaryotic primase (AEP) superfamily. In one embodiment, the archaeal DNA primase belongs to the primase-polymerase (prim-pol) family.

In one embodiment, the archaeal DNA primase is from an archaeon of the Pyrococcus genus. The Pyrococcus genus comprises several species among which, without limitations, Pyrococcus abyssi, Pyrococcus endeavori, Pyrococcus furiosus, Pyrococcus glycovorans, Pyrococcus horikoshii, Pyrococcus kukulkanii, Pyrococcus woesei, and Pyrococcus yayanosii. The Pyrococcus genus also comprises several unclassified strains among which, without limitation, Pyrococcus sp. 12-1, Pyrococcus sp. 121, Pyrococcus sp. 303, Pyrococcus sp. 304, Pyrococcus sp. 312, Pyrococcus sp. 32-4, Pyrococcus sp. 321, Pyrococcus sp. 322, Pyrococcus sp. 323, Pyrococcus sp. 324, Pyrococcus sp. 95-12-1, Pyrococcus sp. AV5, Pyrococcus sp. Ax99-7, Pyrococcus sp. C2, Pyrococcus sp. EX2, Pyrococcus sp. Fla95-Pc, Pyrococcus sp. GB-3A, Pyrococcus sp. GB-D, Pyrococcus sp. GBD, Pyrococcus sp. GI-H, Pyrococcus sp. GI-J, Pyrococcus sp. GIL, Pyrococcus sp. HT3, Pyrococcus sp. JT1, Pyrococcus sp. LMO-A29, Pyrococcus sp. LMO-A30, Pyrococcus sp. LMO-A31, Pyrococcus sp. LMO-A32, Pyrococcus sp. LMO-A33, Pvrococcus sp. LMO-A34, Pvrococcus sp. LMO-A35, Pyrococcus sp. LMO-A36, Pyrococcus sp. LMO-A37, Pyrococcus sp. LMO-A38, Pyrococcus sp. LMO-A39, Pyrococcus sp. LMO-A40, Pyrococcus sp. LMO-A41, Pyrococcus sp. LMO-A42, Pyrococcus sp. M24D13, Pyrococcus sp. MA2.31, Pyrococcus sp. MA2.32, Pyrococcus sp. MA2.34, Pyrococcus sp. MV1019, Pyrococcus sp. MV4, Pyrococcus sp. MV7, Pyrococcus sp. MZ14, Pyrococcus sp. MZ4, Pyrococcus sp. NA2, Pyrococcus sp. NS102-T, Pyrococcus sp. P12.1, Pyrococcus sp. PK 5017, Pyrococcus sp. ST04, Pyrococcus sp. ST700, Pyrococcus sp. Tc-2-70, Pyrococcus sp. Tc95-7C-I, Pyrococcus sp. TC95-7C-S, Pyrococcus sp. Tc95_6, Pyrococcus sp. V211, Pyrococcus sp. V212, Pyrococcus sp. V221, Pyrococcus sp. V222, Pyrococcus sp. V231, Pyrococcus sp. V232, Pyrococcus sp. V61, Pyrococcus sp. V62, Pyrococcus sp. V63, Pyrococcus sp. V72, Pyrococcus sp. V73, Pyrococcus sp. VB112, Pyrococcus sp. VB113, Pyrococcus sp. VB81, Pyrococcus sp. VB82, Pyrococcus sp. VB83, Pyrococcus sp. VB85, Pyrococcus sp. VB86, and Pyrococcus sp. VB93.

In one embodiment, the archaeal DNA primase is selected from the group consisting of Pyrococcus sp. 12-1 DNA primase, Thermococcus sp. CIR10 DNA primase, Thermococcus peptonophilus DNA primase, and Thermococcus celericrescens DNA primase; or a functionally active fragment and/or variant thereof.

In one embodiment, the archaeal DNA primase is Pyrococcus sp. 12-1 DNA primase; or a functionally active fragment and/or variant thereof.

In one embodiment, the amino acid sequence of the Pyrococcus sp. 12-1 DNA primase comprises or consists of SEQ ID NO: 1, which represents the amino acid sequence of the protein “p12-17p” from Pyrococcus sp. 12-1 with NCBI Reference Sequence WP_013087941 version 1 of 2019-05-01.

SEQ ID NO: 1
MRPSDIIIDVYKAIQDHPGAGKLAIELRFYPRPTSEWIIVADIEDKAEE
LHKVLFKNNVLGKKEAYISMALHDFEEVGKKLEKLRELEEERAQKEGRK
PREVTLRNVQGEATGKVHKTVSKYTLTLVVDIDVEEIHKSKVVESEEKA
FELAKRAWDELKPKLEGIGVKPRYVFFTGGGVQLWFVAPGLEPIEVIDR
ASRVIPPVLNAMLPEGYSVDNIFDRARIVRVPLTINYKYKTPDERPLEI
RGRLIEFNDVRTPLGEVLDKLEAYAKEHGISLVTPSQARFIGTVGRYEV
DKGDFRVLAERLVQELAPWFRRVKEKGGSRHHLVNAIAAYIARNTNLTF
EDLAKIWEIVHAELVRLGLEDPNDWSNRYHTIKDVYEKLQAGTFLGTRA
YMLKYLNMMSEEEIVEILRAVKRALFPYLKPVNAGFVSKFVDEPYGRDE
APKKWEDVDEDRRKAVGVFHIRALAFETAEYIYVEDLPKPKTFSIKSHS
EDGKEKLRFNEALWQSFLSWLGVQEGEPFDRVDFENLLIEKFGITEHEL
RSIYFSKVLYLLTPEGMRMPKCIVEFLKELADEGNLSDEKVAHLAHWIK
YFAKPLRHSTTSIMLRAQGKPVDMRMAVWAKIVEFFAEDDDVAKRLVSV
FKKAYEEAEPPFPCFGVKECPFFPGHKGCPFIAPKRNEILAVSLVDVQI
HGTDGIVVVVGGGEGTKEFVLRGRVEWVKVSKKGVRYPIAEWFLDVFAK
EFLALPEPPAWDPGDVTEILKSRARVVKSQLDEFDEYYEHFIDWLRSEN
SRGILPYENADSSHLFIKGKMIGIPPRLAEDFYKIDLGISGKKFKEMLI
RKLGKHFLGKKPVWIRLTSGEKNGVNCYFVSLEWFRDVVGEPNIKEVEA
GEELGEFGFSYEEGEA

In one embodiment, the amino acid sequence of a functionally active fragment of the Pyrococcus sp. 12-1 DNA primase (herein termed “PolpP12_{Δ297-898}”) is as set forth in SEQ ID NO: 2.

SEQ ID NO: 2
MRPSDIIIDVYKAIQDHPGAGKLAIELRFYPRPTSEWIIVADIEDKAEE
LHKVLFKNNVLGKKEAYISMALHDFEEVGKKLEKLRELEEERAQKEGRK
PREVTLRNVQGEATGKVHKTVSKYTLTLVVDIDVEEIHKSKVVESEEKA
FELAKRAWDELKPKLEGIGVKPRYVFFTGGGVQLWFVAPGLEPIEVIDR
ASRVIPPVLNAMLPEGYSVDNIFDRARIVRVPLTINYKYKTPDERPLEI
RGRLIEFNDVRTPLGEVLDKLEAYAKEHGISLVTPSQARFIGTVGRYEV
DK

In one embodiment, the amino acid sequence of a functionally active fragment of the Pyrococcus sp. 12-1 DNA primase (herein termed “PolpP12_{Δ87-92Δ297-898}”) is as set forth in SEQ ID NO: 3.

SEQ ID NO: 3
MRPSDIIIDVYKAIQDHPGAGKLAIELRFYPRPTSEWIIVADIEDKAEE
LHKVLFKNNVLGKKEAYISMALHDFEEVGKKLEKLREQKEGRKPREVTL
RNVQGEATGKVHKTVSKYTLTLVVDIDVEEIHKSKVVESEEKAFELAKR
AWDELKPKLEGIGVKPRYVFFTGGGVQLWFVAPGLEPIEVIDRASRVIP
PVLNAMLPEGYSVDNIFDRARIVRVPLTINYKYKTPDERPLEIRGRLIE
FNDVRTPLGEVLDKLEAYAKEHGISLVTPSQARFIGTVGRYEVDK

In one embodiment, the amino acid sequence of the Thermococcus sp. CIR10 DNA primase comprises or consists of SEQ ID NO: 4, which represents the amino acid sequence of the protein “primase/polymerase” from Thermococcus sp. CIR10 with NCBI Reference Sequence WP_015243587 version 1 of 2016-06-18.

SEQ ID NO: 4
MSGREFKRPSDVIIDIYKVIQDHPEAGRLAIEFRFYPYPTSEWILLNDI
EDKAREIDKVLFKNNILGKKEAYISMAIHDFDEVTKKLEKLQELEHEKA
QKEGRQPKEITLRHVQGEATGKIHTTVSSYTLTLVVDIDVNEIHDSKAV
ESEEKALEVSKRAWEVLKPNLEELGIKPRYVFFTGGGIQLWFVAPEPEN
ISVIDKAAEIIPPVLNTLLPEGYSVDNIFDRARIVRVPFTVNYKYKTPD
GKPLELRGRLLEFNDVRTPLGDILEKLEAYAKGHKISLGSTSRSGKFRG
VAGRYEVKKENFEELAKRLVEELAPWFKKIKERGGSRHHLVNAIAAYIA
RNTNLTEEDLLGKDQEDGTHVVGLWELVHSKLVELGLEDPGDWSNRYHT
IKDVYEKLYAGTTLGTRAYMMKYLNVSEEEAIEILRSVKRALFPYLHPV
NVQVISKFEAKPYSKEEAPTEWEAVDEDRKKAVGIWYIEVLALETANYV
YIEDLSKPGVFYIVEKVKRTVKVGKKEKGVEVDEYHFNPALWQSFLNWL
GIKEGEPIEREELWNLLLEKFDIKDYELRAIYFRKILHLLSPEGMRRPR
CVEEFLRELADEGFLSEDKVRHLAHWIKFYAKPLRHSTTSIMLRAKGKP
VDMRMAVWAKVVEFFAEDEETAQGLIETFREAYEQAEPPFPCFGARECP
FFQEHRGCPFIAPKRDEILAVSLVDVQLHGSDGIVIIVGSEEGTKKFVH
KGKVEWQKQGKSKIKYPVAEWFLDVYAKEFLSLPEAPSWSHEEVTEILK
SRARVVRSQLNEFDEYFDNFIDWLRSENARGIYPYEKADSSHIFIKGNM
IGIPPRLAEDFYRNELGISGRKFKEMLIRELGSYYLGKKAAWIKLSSGQ
HNGVNCYFISLDWFKKIVGEPNIKDIEAEGDIGSGGFNYEEEEGEA

In one embodiment, the amino acid sequence of a functionally active fragment of the Thermococcus sp. CIR10 DNA primase (herein termed “PolpTCIR10_{Δ303-928}”) is as set forth in SEQ ID NO: 5.

SEQ ID NO: 5
MSGREFKRPSDVIIDIYKVIQDHPEAGRLAIEFRFYPYPTSEWILLNDI
EDKAREIDKVLFKNNILGKKEAYISMAIHDFDEVTKKLEKLQELEHEKA
QKEGRQPKEITLRHVQGEATGKIHTTVSSYTLTLVVDIDVNEIHDSKAV
ESEEKALEVSKRAWEVLKPNLEELGIKPRYVFFTGGGIQLWFVAPEPEN
ISVIDKAAEIIPPVLNTLLPEGYSVDNIFDRARIVRVPFTVNYKYKTPD
GKPLELRGRLLEFNDVRTPLGDILEKLEAYAKGHKISLGSTSRSGKFRG
VAGRYEVKK

In one embodiment, the amino acid sequence of the Thermnococcus peptonophilus DNA primase comprises or consists of SEQ ID NO: 6, which represents the amino acid sequence of an “hypothetical protein” from Thermococcus peptonophilus with NCBI Reference Sequence WP_062389070 version 1 of 2016-03-28.

SEQ ID NO: 6
MSELTPGKVLADVYKVIQDHPEAGRLAIELRFYPVIKSEWVLLNDIEDK
ARDIDKVLAKQNLIKGKEAYVSMAIHGFEAVKKKLEKLRESVEEGKVRK
LGLENVQGEAKGKVHPTVSNYTLTLVVDVDIEAVHKLKVVEDVDKVFEK
AKEGWLALKPVFEELGVLPRYVFFTGGGLQLWFVAPKLEDIAVIDRASG
IVPNVLNALLPEGFVVDNIFDRARIVRAPLTVNHKYKAPNGARVGVKGR
LIEFNDVRVSLSEVLDKLEVYAKERGIQLGGQEKVRGGRGFVNVRYVVK
KEELETLALNLADELIPWFKKVKERGGSWHHLVNAIGAYVVRNTNLSLE
DLIGKDNPDGTHVVGLWEIVFQRLVEKSAEDPGDWVNRRNTIKDVYEKH
IAGKPLGTRAYLKKYLPVSDEEVVEILMAVRRALLPFLKEVKKVDSSGF
GVAPYKKTAPRSWDEVDEDRKRATGRWYVWKLAFNTAEYLFTDELPKAG
TFYIDVWMKEGKREWMKRFFNEALFRSFIEDGLGYKYGAPVEREELFER
LVEVFNITDEEVRGIYIDAALSLLSPVGMRTPPCIEEFIMEFAANGSLS
EDKVRHLARWIKLYAKPLKHSTTTTKLVGAGYKVDMRMAVWAKLVEFFA
EDDEVARELVRVFKEEYGAAEPPFTCIGTKTCQFYLNEKMCPFIIPKEK
EILAVSLIDVQRHESDGLVVIVGGDKEVRTFVKKGNVEWVKKTERREKY
PVAEWFIDVFATEYLSVSPDDLDVDLEEVTDILKSRARVVKSRLNELED
MYEKFVEWLKRENAVRGVLPYEKADFNHLFIKGNMIGIPPALAEEFYRF
ELNIKGSEFREMLEKKLGFHYTKKAVKLSVGEKKDVRRCYLVSLEWFRK
VVGEPNVKDVVMAGDIALSGLVYYESGEEVVE

In one embodiment, the amino acid sequence of a functionally active fragment of the Thermococcus peptonophilus DNA primase (herein termed “PolpTpep_{Δ295-914}”) is as set forth in SEQ ID NO: 7.

SEQ ID NO: 7
MSELTPGKVLADVYKVIQDHPEAGRLAIELRFYPVIKSEWVLLNDIEDK
ARDIDKVLAKQNLIKGKEAYVSMAIHGFEAVKKKLEKLRESVEEGKVRK
LGLENVQGEAKGKVHPTVSNYTLTLVVDVDIEAVHKLKVVEDVDKVFEK
AKEGWLALKPVFEELGVLPRYVFFTGGGLQLWFVAPKLEDIAVIDRASG
IVPNVLNALLPEGFVVDNIFDRARIVRAPLTVNHKYKAPNGARVGVKGR
LIEFNDVRVSLSEVLDKLEVYAKERGIQLGGQEKVRGGRGFVNVRYVVK
K

In one embodiment, the amino acid sequence of the Thermococcus celericrescens DNA primase comprises or consists of SEQ ID NO: 8, which represents the amino acid sequence of an “hypothetical protein” from Thermococcus celericrescens with NCBI Reference Sequence WP_058937716 version 1 of 2016-01-06.

SEQ ID NO: 8
MSELTPGKVLADVYKVIQDHPEAGRLAIELRFYPVIKSEWVLLNDIEDK
ARDIDKVLAKKNIINGKEAYVSMAIHDFGAVKKKLEKLREKAEGERARR
IGLENVQGEAKGKVHPTVSNYTLALVVDIDIEEVHKSRVVEDVEAVFER
AKKGWLALRPVFEELGVLPRYVFFTGGGLQIWFVAPELEDIAVIDRASG
IVPGVLNALLPEGFVVDNIFDRARIVRAPLTVNHKYKAPNGAGLGVKGR
LIEFNDVRVSLSEVLDKLEAYAKEKGIQLGGQERASGVRVFGKVRYEVK
KERLETLALNLADELAPWFKKVKERGGSWHHLVNAIGAYIVRNTNLSLE
DLIGKDNPDGTHVVGLWELVFQRLVEKGAEDPSDWLNRRNTIKDVYEKH
IAGKPLGTRAYLKKYLPVSDEEVVEILMAARRALLPFLKGVKRAGSSGF
GVFPYKDTAPRSWNEVEAERKRATGLWYVWSLAFSTAEYIFTDELSKAG
TFFILVKEGKSIKSVFNETLFRSFIEEGLGYKYGMPVRRDELFERLVEV
FNITDEEVRGVYIDAALSLLSPVGMRTPPCIEEFIMEFAANGNLPEDKV
RHLARWIKLYAKPLKHSTTTTKLVGAGYNVDMRMAVWAKLVEFFAEDDE
VAGELVRIFKEEYKEAEPPFTCIGARTCPFYLKDDVAKMCPFIFPKEKE
ILAVSLIDVQRHESDGIVILVGGAREVRTFIKKGKVEWVKRTKKTEKYP
IAEWFIDVFATEYLGVPPDNLDFDLKDVTGILKSRERVVPSQLNEVEDW
YEKFVEWLKRENEVRGVLPYKKADVHHLFIKDNMIGIPPALAEEFYRYE
ANIKPSEFREMLEVKLSIHYKKKAVKLSVGEKKDVRRCYLVSLEWFRKV
VGEPNVKDVVMAGDIALSGLVYYESGEEWE

In one embodiment, the amino acid sequence of a functionally active fragment of the Thermococcus celericrescens DNA primase (herein termed “PolpTcel_{Δ295-913}”) is as set forth in SEQ ID NO: 9.

SEQ ID NO: 9
MSELTPGKVLADVYKVIQDHPEAGRLAIELRFYPVIKSEWVLLNDIEDK
ARDIDKVLAKKNIINGKEAYVSMAIHDFGAVKKKLEKLREKAEGERARR
IGLENVQGEAKGKVHPTVSNYTLALVVDIDIEEVHKSRVVEDVEAVFER
AKKGWLALRPVFEELGVLPRYVFFIGGGLQIWFVAPELEDIAVIDRASG
IVPGVLNALLPEGFVVDNIFDRARIVRAPLTVNHKYKAPNGAGLGVKGR
LIEFNDVRVSLSEVLDKLEAYAKEKGIQLGGQERASGVRVFGKVRYEVK
K

In one embodiment, the isolated functionally active fragment of an archaeal DNA primase or variant thereof according to the present invention comprises or consists of an amino acid sequence selected from the group comprising or consisting of SEQ ID NO: 2, SEQ ID NO: 3, SEQ ID NO: 5, SEQ ID NO: 7, and SEQ ID NO: 9, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 1, SEQ ID NO: 4, SEQ ID NO: 6 and SEQ ID NO: 8.

In one embodiment, the isolated functionally active fragment of an archaeal DNA primase or variant thereof according to the present invention comprises or consists of an amino acid sequence selected from the group comprising or consisting of SEQ ID NO: 2, SEQ ID NO: 5, SEQ ID NO: 7, and SEQ ID NO: 9, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 1, SEQ ID NO: 4, SEQ ID NO: 6 and SEQ ID NO: 8.

In one embodiment, the isolated functionally active fragment of an archaeal DNA primase or variant thereof according to the present invention comprises or consists of the amino acid sequence of SEQ ID NO: 2, or a fragment and/or variant thereof. In one embodiment, the isolated functionally active fragment of an archaeal DNA primase or variant thereof according to the present invention does not consist of the amino acid sequence of SEQ ID NO: 1.

In one embodiment, the isolated functionally active fragment of an archaeal DNA primase or variant thereof according to the present invention comprises or consists of the amino acid sequence of SEQ ID NO: 3, or a fragment and/or variant thereof. In one embodiment, the isolated functionally active fragment of an archaeal DNA primase or variant thereof according to the present invention does not consist of the amino acid sequence of SEQ ID NO: 1.

In one embodiment, a fragment of the isolated functionally active fragment of an archaeal DNA primase or variant thereof according to the present invention comprises or consists of at least 50% contiguous amino acid residues of said isolated functionally active fragment of an archaeal DNA primase or variant thereof, preferably at least 60%, 70%, 80%, 90%, 95% or more contiguous amino acid residues of said isolated functionally active fragment of an archaeal DNA primase or variant thereof.

In one embodiment, a fragment of the isolated functionally active fragment of an archaeal DNA primase or variant thereof according to the present invention remains capable of template-independent terminal nucleotidyl transferase activity, and preferably, is devoid of an ab-initio single-stranded nucleic acid synthesis activity.

In one embodiment, a variant of the isolated functionally active fragment of an archaeal DNA primase or fragment thereof according to the present invention shares at least 70%, preferably at least 75%, 80%, 85%, 90%, 95%, 96%, 97%, 98%, 99% or more sequence identity, preferably local sequence identity with said isolated functionally active fragment of an archaeal DNA primase or fragment thereof.

Sequence identity refers to the number of identical or similar amino acids in a comparison between a test and a reference sequence. Sequence identity can be determined by sequence alignment of protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into number of identical positions account gaps as

$$\frac{number of identical positions}{length of the total aligned sequence} \times 100.$$

A global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

A local alignment is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
(2) a penalty of 3.0 for each gap and an additional 0.10 penalty for each symbol in each gap; and
(3) no penalty for end gaps.

Whether any sequence of a functionally active fragment of an archaeal DNA primase or fragment thereof, and a variant of this sequence, have amino acid sequences that are at least 70%, preferably at least 75%, 80%, 85%, 90%, 95%, 96%, 97%, 98%, 99% or more “identical”, or other similar variations reciting a percent identity, can be determined using known computer algorithms based on local or global alignment (see, e.g., https://en.wikipedia.org/wiki/List_of_sequence_alignment_software, providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi); or LAlign (William Pearson implementing the Huang and Miller algorithm [Huang & Miller, 1991. Adv Appl Math. 12(3):337-57).

Typically, the full-length sequence of each of the compared functionally active fragments of archaeal DNA primases or fragments thereof is aligned across the full-length of each sequence in a global alignment. Local alignment also can be used when the sequences being compared are substantially the same length.

Therefore, the term identity represents a comparison or alignment between a test (the variant) and a reference sequence (the functionally active fragment of an archaeal DNA primase or fragment thereof). In one exemplary embodiment, “at least 70% of sequence identity” refers to percent identities from 70 to 100% relative to the reference sequence. Identity at a level of 70% or more is indicative of the fact that, assuming for exemplification purposes a test and reference sequence length of 100 amino acids are compared, no more than 30 out of 100 amino acids in the test sequence differ from those of the reference sequence. Such differences can be represented as point mutations randomly distributed over the entire length of an amino acid sequence or they can be clustered in one or more locations of varying length up to the maximum allowable, e.g., 30/100 amino acid difference (approximately 70% identity). Differences can also be due to deletions or truncations of amino acid residues. Differences are defined as amino acid substitutions, insertions or deletions. Depending on the length of the compared sequences, at the level of homologies or identities above about 85-90%, the result can be independent of the program and gap parameters set; such high levels of identity can be assessed readily, often without relying on software.

Also encompassed herein are isolated functionally active fragment of an archaeal DNA primase or variant thereof according to the present invention, fused to a processivity factor.

By “processivity factor”, it is meant a polypeptide domain or subdomain which confers sequence-independent nucleic acid interactions, and is associated with the isolated functionally active fragment of an archaeal DNA primase or fragment thereof according to the present invention by covalent or noncovalent interactions. Processivity factors may confer a lower dissociation constant between the archaeal DNA primase and the nucleic acid substrate, allowing for more nucleotide incorporations on average before dissociation of the archaeal DNA primase from the substrate or initiator sequence. Processivity factors function by multiple sequence-independent nucleic acid binding mechanisms: the primary mechanism is electrostatic interaction between the nucleic acid phosphate backbone and the processivity factor; the second is steric interactions between the processivity factor and the minor groove structure of a nucleic acid duplex; the third mechanism is topological restraint, where interactions with the nucleic acid are facilitated by clamp proteins that completely encircle the nucleic acid, with which they associate.

Exemplary sequence-independent nucleic acid binding domains are known in the art, and are traditionally classified according to the preferred nucleic acid substrate, e.g., DNA or RNA and strandedness, such as single-stranded or double-stranded.

Various polypeptide domains have been identified as nucleic acid binders. These polypeptide domains include four general structural topologies known to bind single-stranded DNA: oligonucleotide-binding (OB) folds, K homology (KH) domains, RNA recognition motifs (RRMs), and whirly domains, as described in Dickey et al., 2013. Structure. 21(7):1074-1084.

Oligonucleotide-binding domains (OBDs) are exemplary DNA binding domains structurally conserved in multiple DNA processing proteins. OBDs bind with single-stranded DNA ligands from 3 to 11 nucleotides per OB fold and dissociation constants ranging from low-picomolar to high-micromolar levels. Affinities roughly correlate with the length of single-stranded DNA bound. Some OBDs may confer sequence specific binding, while others are non-sequence specific. Exemplary OBD containing DNA-binding proteins specifically bind single-stranded DNA are so called “single-stranded DNA binding proteins” or “SSBs”. SSB domains are well known to those skilled in the art, as described, e.g., in Keck (Ed.), 2016. Single-stranded DNA binding proteins (Vol. 922, Methods in Molecular Biology). Totowa, N.J.: Humana Press; and Shereda et al., 2008. Crit Rev Biochem Mol Biol. 43(5):289-318. SSBs describe a family of evolved molecular chaperones of single-stranded DNA.

Several exemplary prokaryotic SSBs have been characterized as known to those skilled in the art. These SSBs include, but are not limited to; Escherichia coli SSB (see, e.g., Raghunathan et al., 2000. Nat Struct Biol. 7(8):648-652), Deinococcus radiodurans SSB (see, e.g., Lockhart & DeVeaux, 2013. PLoS One. 8(8):e71651), Sulfolobus solfataricus SSB (see, e.g., Paytubi et al., 2012. Proc Natl Acad Sci USA. 109(7):E398-E405), Thermus thermophillus SSB and Thermus aquaticus SSB (see, e.g., Witte et al., 2008. Biophys J. 94(6):2269-2279), and Deinococcus radiopugnans SSB (see, e.g., Filipkowski et al., 2006. Extremophiles. 10(6):607-614).

In non-eubacterial systems, functional eukaryotic homologs to the prokaryotic SSB protein family are known to those skilled in the art. Replication protein A (RPA) is an exemplary homolog used in DNA replication, recombination and DNA repair in eukaryotes. The RPA heterotrimer is comprised of RPA70, RPA32, RPA14 subunits as described in Iftode et al., 1999. Crit Rev Biochem Mol Biol. 34(3):141-180.

The present invention also relates to a nucleic acid encoding the isolated functionally active fragment of the archaeal DNA primase or variant thereof described above.

It also relates to an expression vector comprising the nucleic acid encoding the isolated functionally active fragment of the archaeal DNA primase or variant thereof described above.

The term “expression vector” refers to a recombinant DNA molecule containing the desired coding nucleic acid sequence and appropriate nucleic acid sequences necessary for the expression of the operably linked coding sequence in a particular host organism.

Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

It also relates to a host cell comprising the expression vector comprising the nucleic acid encoding the isolated functionally active fragment of the archaeal DNA primase or variant thereof described above.

It also relates to a method of producing and purifying the isolated functionally active fragment of the archaeal DNA primase or variant thereof described above.

In one embodiment, the method comprises:
culturing a host cell comprising the expression vector comprising the nucleic acid encoding the isolated functionally active fragment of the archaeal DNA primase or variant thereof described above, under conditions suitable for the expression of said functionally active fragment of the archaeal DNA primase or variant thereof, and
isolating the functionally active fragment of the archaeal DNA primase or variant thereof from said host cell.

This recombinant process can be used for large scale production of the functionally active fragment of the archaeal DNA primase or variant thereof.

In one embodiment, the expressed functionally active fragment of the archaeal DNA primase or variant thereof is further purified.

In a second aspect, the present invention relates to a method for template-independent synthesis of nucleic acids, comprising iteratively contacting an initiator sequence comprising a 3′-end nucleotide with a free 3′-hydroxyl group, with at least one (optionally, selected) nucleoside triphosphate (or a combination of (optionally, selected) nucleoside triphosphates) in the presence of an archaeal DNA primase or a functionally active fragment and/or variant thereof, thereby covalently binding said (optionally, selected) nucleoside triphosphate to the free 3′-hydroxyl group of the 3′-end nucleotide.

In one embodiment, the method of the present invention is a method for template-independent synthesis of nucleic acids with random nucleotide sequence. In one embodiment, the method of the present invention is a method for template-independent, sequence-controlled synthesis of nucleic acids.

References to a “nucleic acid” synthesis method include methods of synthesizing lengths of DNA (deoxyribonucleic acid), RNA (ribonucleic acid), or mixes thereof, wherein a strand of nucleic acid (i.e., an initiator sequence) comprising “n” nucleotides is iteratively extended by adding a further nucleotide “n+1”. The term “nucleic acid” also encompasses nucleic acid analogues, such as, without limitation, xeno nucleic acids (XNA), which are synthetic nucleic acid analogues that have a different sugar backbone and/or outgoing motif than the natural DNAs and RNAs. The term “nucleic acid” hence also encompasses mixed XNA/DNA, mixed XNA/RNA and mixed XNA/DNA/RNA. Examples of XNAs include those described in Schmidt, 2010. Bioessays. 32(4):322-331 and Nie et al., 2020. Molecules. 25(15):E3483, the content of which is herein incorporated by reference. Some examples include, but are not limited to, 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), and fluoro arabino nucleic acid (FANA) (Schmidt, 2008. Syst Synth Biol. 2(1-2):1-6; Ran et al., 2009. Nat Nanotechnol. 4(10):6; Kershner et al., 2009. Nat Nanotechnol. 4(9):557-61; Marliere, 2009. Syst Synth Biol. 3(1-4):77-84; Torres et al., 2003. Microbiology. 149(Pt 12):3595-601; Vastmans et al., 2001. Nucleic Acids Res. 29(15):3154-63; Ichida et al., 2005. Nucleic Acids Res. 33(16):5219-25; Kempeneers et al., 2005. Nucleic Acids Res. 33(12):3828-36; Loakes et al., 2009. J Am Chem Soc. 131(41):14827-37).

References to a “template-independent” nucleic acid synthesis method illustrate those methods of nucleic acid synthesis which do not require a template nucleic acid strand, i.e., the nucleic acid is synthesized de novo.

References to a “sequence-controlled” nucleic acid synthesis method illustrate those methods of nucleic acid synthesis which allow the specific addition of selected nucleotides “n+1” to a strand of nucleic acid (i.e., an initiator sequence) comprising “n” nucleotides, i.e., the synthesized nucleic acid has a defined—by contrast to random —nucleotide sequence.

In one embodiment, the archaeal DNA primase or the functionally active fragment and/or variant thereof belongs to the archaeo-eukaryotic primase (AEP) superfamily.

In one embodiment, the archaeal DNA primase or the functionally active fragment and/or variant thereof is from an archaeon of the Thermococcales order.

In one embodiment, the archaeal DNA primase is from an archaeon of the Pyrococcus genus.

In one embodiment, the archaeal DNA primase or the functionally active fragment and/or variant thereof belongs to the primase-polymerase (prim-pol) family (also termed “PolpTN2-like family” by Kazlauskas et al., 2018. J Mol Biol. 430(5):737-750).

In one embodiment, the archaeal DNA primase or the functionally active fragment and/or variant thereof comprises or consists of the primase domain of an archaeal DNA primase belonging to the primase-polymerase (prim-pol) family (as shown by Kazlauskas et al., 2018. J Mol Biol. 430(5):737-750 in their FIG. 6).

In one embodiment, the archaeal DNA primase is selected from the group consisting of Pyrococcus sp. 12-1 DNA primase, Thermococcus sp. CIR10 DNA primase, Thermococcus peptonophilus DNA primase, Thermococcus celericrescens DNA primase, and Thermococcus nautili sp. 30-1 DNA primase; or a functionally active fragment and/or variant thereof, as described hereinabove.

In one embodiment, the archaeal DNA primase is selected from the group consisting of Pyrococcus sp. 12-1 DNA primase, Thermococcus sp. CIR10 DNA primase, Thermococcus peptonophilus DNA primase, and Thermococcus celericrescens DNA primase; or a functionally active fragment and/or variant thereof, as described hereinabove.

In one embodiment, the archaeal DNA primase is Pyrococcus sp. 12-1 DNA primase; or a functionally active fragment and/or variant thereof, as described hereinabove.

In one embodiment, the amino acid sequence of the Pyrococcus sp. 12-1 DNA primase comprises or consists of SEQ ID NO: 1, as described hereinabove.

In one embodiment, the amino acid sequence of a functionally active fragment of the Pyrococcus sp. 12-1 DNA primase (herein termed “PolpP12_{Δ297-898}”) is as set forth in SEQ ID NO: 2.

In one embodiment, the amino acid sequence of a functionally active fragment of the Pyrococcus sp. 12-1 DNA primase (herein termed “PolpP12A87-92_{Δ297-898}”) is as set forth in SEQ ID NO: 3.

In one embodiment, the amino acid sequence of the Thermococcus sp. CIR10 DNA primase comprises or consists of SEQ ID NO: 4, as described hereinabove.

In one embodiment, the amino acid sequence of a functionally active fragment of the Thermococcus sp. CIR10 DNA primase is as set forth in SEQ ID NO: 5.

In one embodiment, the amino acid sequence of the Thermococcus peptonophilus DNA primase comprises or consists of SEQ ID NO: 6, as described hereinabove.

In one embodiment, the amino acid sequence of a functionally active fragment of the Thermococcus peptonophilus DNA primase is as set forth in SEQ ID NO: 7.

In one embodiment, the amino acid sequence of the Thermococcus celericrescens DNA primase comprises or consists of SEQ ID NO: 8, as described hereinabove.

In one embodiment, the amino acid sequence of a functionally active fragment of the Thermococcus celericrescens DNA primase is as set forth in SEQ ID NO: 9.

In one embodiment, the amino acid sequence of the Thermococcus nautili sp. 30-1 DNA primase comprises or consists of SEQ ID NO: 10, which represents the amino acid sequence of the protein “tn2-12p” from Thermococcus nautili sp. 30-1 with NCBI Reference Sequence WP_013087990 version 1 of 2019-05-01.

SEQ ID NO: 10
MSSLRPSSIIIDIYKVIQDHPDSSRWAIELRHLPKPISSEWIHITDIEE
RASEIDKVLIKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTIIK
DEDGTERVVPTQVKGRVIEFNDVRISLTEFLDRLEAYAKEKGIPLEKPT
KRKFLELASKRYEVTSSNFEALAERLFTELRPWWEIAKEKGWSRHHLTM
GIATYILRNTNLTPEQLIGSENSPGLWELVFAKLVEAGLEDPDDWKNRA
STIRDAYKKIESGKKVATKAYLRKYIEGLSEEDAVQILLSVKRALLPYL
KAVDVKRISKYSARPYEITEEIPKSWEDIDENRKKATGVWYIDFLALET
ANDIYFEDLPKPPVFYIRFVEKNKEKFKLNETLYHSFLTWLGITEGEPL
DRTELIDLLVEKFGFTVEDLKAIYYRKILTLLKPEGMRTPKCIQEFLFE
LATEGNLPEDKIRHLAHWVKFYARPLRHSTTSVLLKGRGKPVDMRLAIW
AKVVEFFAEDDEVAEELITTFKKAYMGAEPPFPCIGAESCPFYPDHRAC
PFIVPKRKEVLPVSIVDVQLHGSDGIVVLVGGPTEVTAFTLEGKVEWIK
TTKKTVKYPIAEWFLDRFAKEYLSLPEAPSWWKLEEVTEILKSRARVVK
SQFDKFEDYLEQFIEWLQKENSRRGILPYEKADENHLFIKGEWVGIPPG
FAREFYSGELLIGGPTFRRMLEQKLGKDYRKMSAKIHLNTGLKDKRNCY
FVSVEWFRKHVGEPNIQEITSEGDVSFDGLSYDDEEEGVVG

In one embodiment, the amino acid sequence of a functionally active fragment of the Thermococcus nautili sp. 30-1 DNA primase (herein termed “PolpTN2_{Δ311-923}”) is as set forth in SEQ ID NO: 11.

SEQ ID NO: 11
MSSLRPSSIIIDIYKVIQDHPDSSRWAIELRHLPKPISSEWIHITDIEE
RASEIDKVLIKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTIIK
DEDGTERVVPTQVKGRVIEFNDVRISLTEFLDRLEAYAKEKGIPLEKPT
KRKFLELASKRYEVTS

In one embodiment, the archaeal DNA primase or functionally active fragment and/or variant thereof comprises or consists of an amino acid sequence selected from the group comprising or consisting of SEQ ID NO: 1, SEQ ID NO: 4, SEQ ID NO: 6, SEQ ID NO: 8 and SEQ ID NO: 10; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 3, SEQ ID NO: 5, SEQ ID NO: 7, SEQ ID NO: 9 and SEQ ID NO: 11; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 5, SEQ ID NO: 7, SEQ ID NO: 9 and SEQ ID NO: 11; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 1, SEQ ID NO: 4, SEQ ID NO: 6 and SEQ ID NO: 8; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 3, SEQ ID NO: 5, SEQ ID NO: 7 and SEQ ID NO: 9; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 5, SEQ ID NO: 7 and SEQ ID NO: 9; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 2; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 3; or a functionally active fragment and/or variant thereof.

In one embodiment, the archaeal DNA primase or functionally active fragment and/or variant thereof comprises or consists of the amino acid sequence set forth in SEQ ID NO: 4; or a functionally active fragment and/or variant thereof. In one embodiment, the archaeal DNA primase or functionally active fragment and/or variant thereof comprises or consists of the amino acid sequence set forth in SEQ ID NO: 5; or a functionally active fragment and/or variant thereof.

In one embodiment, the archaeal DNA primase or functionally active fragment and/or variant thereof comprises or consists of the amino acid sequence set forth in SEQ ID NO: 6; or a functionally active fragment and/or variant thereof. In one embodiment, the archaeal DNA primase or functionally active fragment and/or variant thereof comprises or consists of the amino acid sequence set forth in SEQ ID NO: 7; or a functionally active fragment and/or variant thereof.

In one embodiment, the archaeal DNA primase or functionally active fragment and/or variant thereof comprises or consists of the amino acid sequence set forth in SEQ ID NO: 8; or a functionally active fragment and/or variant thereof. In one embodiment, the archaeal DNA primase or functionally active fragment and/or variant thereof comprises or consists of the amino acid sequence set forth in SEQ ID NO: 9; or a functionally active fragment and/or variant thereof.

In one embodiment, the archaeal DNA primase or functionally active fragment and/or variant thereof comprises or consists of the amino acid sequence set forth in SEQ ID NO: 10; or a functionally active fragment and/or variant thereof. In one embodiment, the archaeal DNA primase or functionally active fragment and/or variant thereof comprises or consists of the amino acid sequence set forth in SEQ ID NO: 11; or a functionally active fragment and/or variant thereof.

In one embodiment, a fragment of the archaeal DNA primase or functionally active fragment and/or variant thereof comprises or consists of at least 50% of contiguous amino acid residues of said archaeal DNA primase or functionally active fragment and/or variant thereof, preferably at least 60%, 70%, 80%, 90%, 95% or more of contiguous amino acid residues of said archaeal DNA primase or functionally active fragment and/or variant thereof.

In one embodiment, a fragment of the archaeal DNA primase or functionally active fragment and/or variant thereof remains capable of template-independent terminal nucleotidyl transferase activity, and preferably, is devoid of an ab-initio single-stranded nucleic acid synthesis activity.

In one embodiment, the archaeal DNA primase or the functionally active fragment and/or variant thereof is fused to a processivity factor.

Processivity factors have been described hereinabove, which description applies mutatis mutandis to the archaeal DNA primase or the functionally active fragment and/or variant thereof.

By “initiator sequence” or “primer”, it is meant a short oligonucleotide with a free 3′-end onto which a (optionally, selected) nucleoside triphosphate can be covalently bound, i.e., the nucleic acid will be synthesized from the 3′-end of the initiator sequence.

In one embodiment, the initiator sequence is a DNA initiator sequence. In one embodiment, the initiator sequence is an RNA initiator sequence. In one embodiment, the initiator sequence is a XNA initiator sequence. In one embodiment, the initiator sequence is a mixed DNA/RNA initiator sequence. In one embodiment, the initiator sequence is a mixed XNA/DNA initiator sequence. In one embodiment, the initiator sequence is a mixed XNA/RNA initiator sequence. In one embodiment, the initiator sequence is a mixed XNA/DNA/RNA initiator sequence.

In one embodiment, the initiator sequence has a length ranging from 2 to 50 nucleotides. In one embodiment, the initiator sequence comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In one embodiment, the initiator sequence is single-stranded. In one embodiment, the initiator sequence is double-stranded. In the latter embodiment, it will be understood by the one skilled in the art that a 3′-overhang (i.e., a free 3′-end) is preferable for a more efficient binding of the (optionally, selected) nucleoside triphosphate.

In one embodiment, the initiator sequence may be immobilized onto a support. In particular, the use of supports allows to easily filter, wash and/or elute reagents and by-products, without washing away the synthesized nucleic acid.

Suitable examples of supports include, but are not limited to, beads, slides, chips, particles, strands, gels, sheets, tubing, spheres, containers, capillaries, pads, slices, films, culture dishes, microtiter plates, and the like. Exemplary materials that can be used for such supports include, but are not limited to, acrylics, carbon (e.g., graphite, carbon-fiber), cellulose (e.g., cellulose acetate), ceramics, controlled-pore glass, cross-linked polysaccharides (e.g., agarose, SEPHAROSE™ or alginate), gels, glass (e.g., modified or functionalized glass), gold (e.g., atomically smooth Au(111)), graphite, inorganic glasses, inorganic polymers, latex, metal oxides (e.g., SiO₂, TiO₂, stainless steel), metalloids, metals (e.g., atomically smooth Au(111)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™; or a mixture thereof.

In one embodiment, the initiator sequence is immobilized onto a support via a reversible interacting moiety, such as, e.g., a chemically-cleavable linker, an enzymatically-cleavable linker, or any other suitable means.

It is thus conceivable that the synthetized nucleic acid be ultimately cleaved from the support and, e.g., amplified using the initiator sequence as a template. The initiator sequence could therefore contain an appropriate forward primer sequence, and an appropriate reverse primer could be synthesized.

Additionally or alternatively, the immobilized initiator sequence may contain a restriction site.

It is thus conceivable that the synthetized nucleic acid be ultimately cleaved from the support using a restriction enzyme.

Additionally or alternatively, the immobilized initiator sequence may contain a uridine.

It is thus conceivable that the synthetized nucleic acid be ultimately cleaved from the support using (1) a uracil-DNA glycosylase (UDG) to generate an abasic site, and (2) an apurinic/apyrimidinic (AP) site endonuclease to cleave the synthetized nucleic acid at the abasic site.

Additionally or alternatively, the immobilized initiator sequence may contain a sequence complementary to a small interfering nucleic acid guide sequence.

It is thus conceivable that the synthetized nucleic acid be ultimately cleaved from the support using a small interfering nucleic acid guide sequence to target an endonuclease such as, e.g., Argonaute, to the immobilized initiator sequence and cleave the synthetized nucleic acid.

By “nucleoside triphosphate” or “NTP”, it is referred herein to a molecule containing a nitrogenous base bound to a 5-carbon sugar (typically, either ribose or deoxyribose), with three phosphate groups bound to the sugar at position 5. The term “nucleoside triphosphate” also encompasses nucleoside triphosphate analogues, such as, nucleoside triphosphates with a different sugar and/or a different nitrogenous base than the natural NTPs, as well as nucleoside triphosphates with a modified 2′-OH, 3′-OH and/or 5′-triphosphate position. In particular, nucleoside triphosphate analogues include those useful for the synthesis of xeno nucleic acids (XNA), as defined hereinabove. Non-limiting examples of such synthetic nucleoside triphosphate analogues are given in FIG. 4 of Chakravarthy et al., 2017 (Theranostics. 7(16):3933-3947), the content of which Figure is herein incorporated by reference. Further non-limiting examples of such synthetic nucleoside triphosphate analogues are given in [0250] to [0280] of US 20090286696, the content of which paragraphs is herein incorporated by reference. A nucleoside triphosphate containing a deoxyribose is typically referred to as deoxynucle oside triphosphate and abbreviated as dNTP. Consistently, a nucleoside triphosphate containing a ribose is typically referred to as ribonucleoside triphosphate and abbreviated as rNTP.

Examples of deoxynucleoside triphosphates include, but are not limited to, deoxyadenosine triphosphate (dATP), deoxyguanosine triphosphate (dGTP), deoxycytidine triphosphate (dCTP), and deoxythymidine triphosphate (dTTP). Further examples of deoxynucleoside triphosphates include deoxyuridine triphosphate (dUTP), deoxyinosine triphosphate (dITP), and deoxyxanthosine triphosphate (dXTP).

Examples of ribonucleoside triphosphates include, but are not limited to, adenosine triphosphate (ATP), guanosine triphosphate (GTP), cytidine triphosphate (CTP) and uridine triphosphate (UTP). Further examples of nucleoside triphosphates include N-methyladenosine triphosphate (m⁶ATP), 5-methyluridine triphosphate (m⁵UTP), 5-methylcytidine triphosphate (m⁵CTP), pseudouridine triphosphate (ψUTP), inosine triphosphate (ITP), xanthosine triphosphate (XTP), and wybutosine triphosphate (yWTP).

Other types of nucleosides may be bound to three phosphates to form nucleoside triphosphates, such as naturally occurring modified nucleosides and artificial nucleosides.

By “selected” with reference to nucleoside triphosphates, it is meant a nucleoside triphosphate or a combination of nucleoside triphosphates purposely chosen among the various possibilities of nucleoside triphosphates, including, but not limited to those described above, with the idea of synthetizing either (1) a nucleic acid with a random sequence or (2) a nucleic acid with a defined nucleotide sequence.

By “combination of nucleoside triphosphates”, it is meant a mix of at least two different nucleoside triphosphates.

In one embodiment, the method of the present invention is a method for template-independent synthesis of nucleic acids with random nucleotide sequence, which comprises—optionally iteratively—contacting an initiator sequence comprising a 3′-end nucleotide with a free 3′-hydroxyl group, with a (optionally, selected) combination of nucleoside triphosphates in the presence of an archaeal DNA primase or a functionally active fragment and/or variant thereof, thereby covalently and randomly binding said combination of (optionally, selected) nucleoside triphosphates to the free 3′-hydroxyl group of the 3′-end nucleotide.

In this embodiment, the (optionally, selected) combination of nucleoside triphosphates does not comprise terminating nucleoside triphosphates.

In one embodiment, the method of the present invention is a method for template-independent, sequence-controlled synthesis of nucleic acids, which comprises iteratively contacting an initiator sequence comprising a 3′-end nucleotide with a free 3′-hydroxyl group of a nucleotide with a selected terminating nucleoside triphosphate in the presence of an archaeal DNA primase or a functionally active fragment and/or variant thereof, thereby covalently binding said selected terminating nucleoside triphosphate to the free 3′-hydroxyl group of the 3′-end nucleotide.

In the latter embodiment of sequence-controlled synthesis of nucleic acids, the 3′-hydroxyl group of a 3′-end nucleotide is contacted with a selected terminating nucleoside triphosphate.

By “terminating nucleoside triphosphate”, also sometimes termed “3′-blocked nucleoside triphosphates” or “3′-protected nucleoside triphosphates”, it is referred to nucleoside triphosphate which have an additional group (hereafter, “3′-blocking group” or “3′-protecting group”) on their 3′-end (i.e., at position 3 of their 5-carbon sugar), for the purpose of preventing further, undesired, addition of nucleoside triphosphates after specific addition of the selected nucleotide (n+1) to a strand of nucleic acid (n).

In one embodiment, the 3′-blocking group may be reversible (can be removed from the nucleoside triphosphate) or irreversible (cannot be removed from the nucleoside triphosphate), i.e., the terminating nucleoside triphosphate may be a reversible terminating nucleoside triphosphate or a non-reversible terminating nucleoside triphosphate.

In one embodiment, the 3′-blocking group is reversible, and removal of the 3′-blocking group from the nucleoside triphosphate (e.g., using a cleaving agent) allows the addition of further nucleoside triphosphate to the synthetized nucleic acid.

Examples of reversible 3′-blocking groups include, but are not limited t, methyl, methoxy, oxime, 2-nitrobenzyl, 2-cyanoethyl, allyl, amine, aminoxy, azidomethyl, tert-butoxy ethoxy (TBE), propargyl, acetyl, quinone, coumarin, aminophenol derivative, ketal, N-methyl-anthraniloyl, and the like.

In the context of the present invention, the term “cleaving agent” refers to any chemical, biological or physical agent which is able to remove (or cleave) a reversible 3′-blocking group from a reversible terminating nucleoside triphosphate.

In one embodiment, the cleaving agent is a chemical cleaving agent. In one embodiment, the cleaving agent is an enzymatic cleaving agent. In one embodiment, the cleaving agent is a physical cleaving agent.

It will be understood by the one skilled in the art that the selection of a cleaving agent is dependent on the type of 3′-blocking group used. For instance, tris(2-carboxyethyl)phosphine (TCEP) can be used to cleave a 3′-O-azidomethyl group, palladium complexes can be used to cleave a 3′-O-allyl group, sodium nitrite can be used to cleave a 3′-aminoxy group, and UV light can be used to cleave a 3′-O-nitrobenzyl group.

In one embodiment, the cleaving agent may be used in conjunction with a cleavage solution comprising a denaturant (such as, e.g., urea, guanidinium chloride, formamide or betaine). In particular, adding a denaturant provides the advantage of disrupting any undesirable secondary structures in the synthetized nucleic acid. The cleavage solution may further comprise one or more buffers, which will be dependent on the exact cleavage chemistry and cleaving agent used.

In one embodiment, the 3′-blocking group is irreversible, and addition of a non-reversible terminating nucleoside triphosphate to the synthetized nucleic acid terminates the synthesis. Such irreversible 3′-blocking groups may be useful, e.g., as fluorophores, labels, tags, etc.

Example of irreversible 3′-blocking groups include, but are not limited to, fluorophores, such as, e.g., methoxycoumarin, dansyl, pyrene, Alexa Fluor 350, AMCA, Marina Blue dye, dapoxyl dye, dialkylaminocoumarin, bimane, hydroxycoumarin, Cascade Blue dye, Pacific Orange dye, Alexa Fluor 405, Cascade Yellow dye, Pacific Blue dye, PyMPO, Alexa Fluor 430, NBD, QSY 35, fluorescein, Alexa Fluor 488, Oregon Green 488, BODIPY 493/503, rhodamine green dye, BODIPY FL, 2′,7′-dichlorofluorescein, Oregon Green 514, Alexa Fluor 514, 4′,5′-dichloro-2′,7′-dimethoxyfluorescein (JOE), eosin, rhodamine 6G, BODIPY R6G, Alexa Fluor 532, BODIPY 530/550, BODIPY TMR, Alexa Fluor 555, tetramethylrhodamine (TMR), Alexa Fluor 546, BODIPY 558/568, QSY 7, QSY 9, BODIPY 564/570, lissamine rhodamine B, rhodamine red dye, BODIPY 576/589, Alexa Fluor 568, X-rhodamine, BODIPY 581/591, BODIPY TR, Alexa Fluor 594, Texas Red dye, naphthofluorescein, Alexa Fluor 610, BODIPY 630/650, malachite green, Alexa Fluor 633, Alexa Fluor 635, BODIPY 650/665, Alexa Fluor 647, QSY 21, Alexa Fluor 660, Alexa Fluor 680, Alexa Fluor 700, Alexa Fluor 750, Alexa Fluor 790, and the like.

Further examples of irreversible 3′-blocking groups include, but are not limited to, biotin or desthiobiotin groups.

In any one of the above embodiments, the nucleoside triphosphate is a 2′-protected nucleoside triphosphate.

By “2′-protected nucleoside triphosphate” it is referred to nucleoside triphosphates which have an additional group (hereafter, “2′-protecting group”) on their 2′-end (i.e., at position 2 of their 5-carbon sugar). A particular—although not the sole—purpose of such 2′-protecting groups is to protect the reactive 2′-hydroxyl group in the specific case of ribonucleotide triphosphates.

Any 3′-blocking groups described above, whether reversible or irreversible, are also suitable to serve as 2′-protecting groups.

Additionally, any 3′-blocking groups described above, whether reversible or irreversible, can further be added at any position of the nucleoside triphosphates, whether on their 5-carbon sugar moiety and/or on their nitrogenous base.

In one embodiment, the method for template-independent synthesis of nucleic acids comprises the following steps:
a) providing an initiator sequence comprising a 3′-end nucleotide with a free 3′-hydroxyl group;
b) contacting said 3′-end nucleotide with a (optionally, selected) nucleoside triphosphate (or a combination of (optionally, selected) nucleoside triphosphates) in the presence of an archaeal DNA primase or a functionally active fragment and/or variant thereof;

thereby covalently binding said (optionally, selected) nucleoside triphosphate to the free 3′-hydroxyl group of the 3′-end nucleotide.

In one embodiment, the method according to the present invention is for template-independent synthesis of nucleic acids with a random sequence, and it comprises the following steps:
a) providing an initiator sequence comprising a 3′-end nucleotide with a free 3′-hydroxyl group;
b) contacting said 3′-end nucleotide with a combination of (optionally, selected) nucleoside triphosphates in the presence of an archaeal DNA primase or a functionally active fragment and/or variant thereof;

thereby randomly covalently binding said combination of (optionally, selected) nucleoside triphosphates to the free 3′-hydroxyl group of the 3′-end nucleotide.

In one embodiment, the method according to the present invention is for template-independent, sequence-controlled synthesis of nucleic acids, and it comprises the following steps:
a) providing an initiator sequence comprising a 3′-end nucleotide with a free 3′-hydroxyl group;
b) contacting said 3′-end nucleotide with a selected reversibly terminating nucleoside triphosphate in the presence of an archaeal DNA primase or a functionally active fragment and/or variant thereof,

thereby covalently binding said selected reversibly terminating nucleoside triphosphate to the free 3′-hydroxyl group of the 3′-end nucleotide;
c) applying a w ashing solution to remove all reagents, in particular to remove unbound reversibly terminating nucleoside triphosphates;
d) cleaving the reversible 3′-blocking group of the covalently bound terminating nucleoside triphosphate in the presence of a cleaving agent;

thereby obtaining a nucleotide with a free 3′-hydroxyl group;
e) optionally, applying a washing solution to remove all reagents, in particular to remove the cleaving agent,
f) optionally, reiterating steps b) to e) multiple times to synthetize a nucleic acid of desired length and nucleotide sequence.

In one embodiment, more than 1 nucleoside triphosphate is added to the 3′-end nucleotide with a free 3′-hydroxyl group, such as, more than 2, 5, 10, 20, 30, 40, 50, 60, 70, 80, 90, 100, 200, 300, 400, 500, 600, 700, 800, 900, 1000, 1250, 1500, 1750, 2000, 2250, 2500, 2750, 3000, 4000, 5000, 6000, 7000, 8000, 9000, 10000 or even more nucleoside triphosphates are added to the 3′-end nucleotide with a free 3′-hydroxyl group by reiterating steps b) to e) as many times.

In one embodiment, the method for template-independent synthesis of nucleic acids according to the present invention is carried out in the presence of one or more buffers (e.g., Tris or cacodylate) and/or one or more salts (e.g., Na⁺, K⁺, Mg²⁺, Mn²⁺, Cu²⁺, Zn²⁺, Co²⁺, etc., all with appropriate counterions, such as Cl⁻).

In one embodiment, the method for template-independent synthesis of nucleic acids according to the present invention is carried out in the presence of one or more divalent cations (e.g., Mg²⁺, Mn²⁺, Co²⁺, etc., all with appropriate counterions, such as Cl⁻), preferably in the presence of Mn²⁺.

In one embodiment, the method for template-independent synthesis of nucleic acids according to the present invention is carried out at a temperature ranging from about from about 60° C. to about 95° C. In one embodiment, the method for template-independent synthesis of nucleic acids according to the present invention is carried out at a temperature of about 60° C., 65° C., 70° C., 75° C., 80° C., 85° C., 90° C. or 95° C.

In one embodiment, the archaeal DNA primase or functionally active fragment and/or variant thereof may be used in the method for template-independent synthesis of nucleic acids according to the present invention for producing synthetic homo- and heteropolymers. One skilled in the art is familiar with means and methods for producing synthetic homo- and heteropolymers, described in, e.g., Bollum, 1974 (In Boyer [Ed.], The enzymes [3^{rd} ed., Vol. 10, pp. 145-171]. New York, N.Y.: Academic Press), the content of which is incorporated herein by reference.

In one embodiment, the archaeal DNA primase or functionally active fragment and/or variant thereof may be used in the method for template-independent synthesis of nucleic acids according to the present invention for homopolymeric tailing of any type of 3′-OH terminus. One skilled in the art is familiar with means and methods for homopolymeric tailing, described in, e.g., Deng & Wu, 1983 (Methods Enzymol. 100:96-116) and Eschenfeldt et al., 1987 (Methods Enzymol. 152:337-342), the content of which is incorporated herein by reference.

In one embodiment, the archaeal DNA primase or functionally active fragment and/or variant thereof may be used in the method for template-independent synthesis of nucleic acids according to the present invention for oligonucleotide, DNA, and RNA labeling. One skilled in the art is familiar with means and methods for labelling, described in, e.g., Deng & Wu, 1983 (Methods Enzymol. 100:96-116), Tu & Cohen, 1980 (Gene. 10(2):177-183), Vincent et al., 1982 (Nucleic Acids Res. 10(21):6787-6796), Kumar et al., 1988 (Anal Biochem. 169(2):376-382), Gaastra & Klemm, 1984 (In Walker et al. [Eds.], Nucleic acids [Vol. 2, Methods in molecular biology, pp. 269-271]. Clifton, N.J.: Humana Press), Igloi & Schiefermayr, 1993 (Biotechniques. 15(3):486-497) and Winz et al., 2015 (Nucleic Acids Res. 43(17):e110), the content of which is incorporated herein by reference.

In one embodiment, the archaeal DNA primase or functionally active fragment and/or variant thereof may be used in the method for template-independent synthesis of nucleic acids according to the present invention for 5′-RACE (Rapid Amplification of cDNA Ends). One skilled in the art is familiar with means and methods for 5′-RACE, described in, e.g., Scotto-Lavino et al., 2006 (Nat Protoc. 1(6):2555-62), the content of which is incorporated herein by reference.

In one embodiment, the archaeal DNA primase or functionally active fragment and/or variant thereof may be used in the method for template-independent synthesis of nucleic acids according to the present invention for in situ localization of apoptosis, such as TUNEL (terminal deoxynucleotidyl transferase dUTP nick end labeling) assay. One skilled in the art is familiar with means and methods for in situ localization of apoptosis such as TUNEL assay, described in, e.g., Gorczyca et al., 1993 (Cancer Res. 53(8):1945-1951) and Lebon et al., 2015 (Anal Biochem. 480:37-41), the content of which is incorporated herein by reference.

In a third aspect, the present invention relates to a system for template-independent synthesis of nucleic acids, comprising:
an initiator sequence comprising a 3′-end nucleotide with a free 3′-hydroxyl group, optionally wherein said initiator sequence is immobilized onto a support;
nucleoside triphosphates; and
an archaeal DNA primase or a functionally active fragment and/or variant thereof.

In one embodiment, the system is suitable for template-independent synthesis of nucleic acids with a random sequence, and it comprises:
an initiator sequence comprising a 3′-end nucleotide with a free 3′-hydroxyl group, optionally wherein said initiator sequence is immobilized onto a support;
a combination of (optionally, selected) nucleoside triphosphates, wherein said (optionally, selected) nucleoside triphosphates are not terminating nucleoside triphosphates; and
an archaeal DNA primase or a functionally active fragment and/or variant thereof.

In one embodiment, the system is suitable for template-independent, sequence-controlled synthesis of nucleic acids, and it comprises:
an initiator sequence comprising a 3′-end nucleotide with a free 3′-hydroxyl group, optionally wherein said initiator sequence is immobilized onto a support;
reversibly terminating selected nucleoside triphosphates, wherein different nucleoside triphosphates are not combined together in a same vial;
a cleaving agent; and
an archaeal DNA primase or a functionally active fragment and/or variant thereof.

In a fourth aspect, the present invention relates to a kit comprising:
an initiator sequence comprising a 3′-end nucleotide with a free 3′-hydroxyl group, optionally wherein said initiator sequence is immobilized onto a support;
nucleoside triphosphates; and
an archaeal DNA primase or a functionally active fragment and/or variant thereof.

In one embodiment, the kit comprises:
an initiator sequence comprising a 3′-end nucleotide with a free 3′-hydroxyl group, optionally wherein said initiator sequence is immobilized onto a support;
a combination of (optionally, selected) nucleoside triphosphates, wherein said (optionally, selected) nucleoside triphosphates are not terminating nucleoside triphosphates; and
an archaeal DNA primase or a functionally active fragment and/or variant thereof.

In one embodiment, the kit comprises:
an initiator sequence comprising a 3′-end nucleotide with a free 3′-hydroxyl group, optionally wherein said initiator sequence is immobilized onto a support;
reversibly terminating selected nucleoside triphosphates, wherein different nucleoside triphosphates are not combined together in a same vial;
a cleaving agent; and
an archaeal DNA primase or a functionally active fragment and/or variant thereof.

### BRIEF DESCRIPTION OF THE DRAWINGS

FIG. 1 is a phylogenetic tree generated using BLOSUM62 with average distance of ten archaeal DNA primases or functionally active fragments thereof, from Pyrococcus sp. 12-1 (fragment PolpP12_{Δ297-898}), Thermococcus sp. CIR10 (fragment PolpTCIR10_{Δ303-928}), Thermococcus peptonophilus (fragment PolpTpep_{Δ295-914}), Thermococcus celericrescens (fragment PolpTcele_{Δ295-913}), Pyrococcus furiosus (full-length), Thermococcus kodakarensis (full-length), Saccharolobus solfataricus (full-length), Pyrococcus horikoshii (full-length), Archaeoglobus fulgidus (full-length) and Thermococcus nautili sp. 30-1 (fragment PolpTN2A311-923).

FIG. 2 is a photograph of an electrophoresis gel (SDS-PAGE) showing the purification of PolpTN2_{Δ311-923} (middle lane) and PolpP12_{Δ297-898} (right lane). MW ladder: molecular weight ladder (left lane).

FIG. 3 is a photograph of a 15% urea-PAGE showing a template-independent nucleic acid synthesis assay using PolpP12_{Δ297-898} [PolpP12_{Δ}] or PolpTN2_{Δ311-923} [PolpTN2_{Δ}], at 60° C., 70° C. or 80° C. [a]: initiator sequence only, no enzyme, no dNTP; [b]: initiator sequence+enzyme, no dNTP; [c]: initiator sequence+enzyme+dNTP mix (unprotected).

FIGS. 4A-C are a set of three photographs of a 1.5% agarose gel electrophoresis showing a template-independent nucleic acid synthesis assay using PolpP12_{Δ297-898} or PolpTN2_{Δ311-923} at 70° C., 80° C., 90° C. and 100° C., in comparison with a negative control performed at 70° C. without enzyme [No Enzyme]. dsDNA LF Ladder: SmartLadder 200 to 10000 bp (Eurogentec).

FIG. 4A: red channel, Cy5 fluorescence at 675 nm;

FIG. 4B: green channel, Sybr green II fluorescence at 520 nm;

FIG. 4C: merge of red and green channels.

FIGS. 5A-C are a set of three photographs of a 1.5% agarose gel electrophoresis showing a template-independent nucleic acid synthesis assay using PolpP12_{Δ297-898} or PolpTN2_{Δ311-923} and carried out in the presence or in the absence of dNTPs and/or the initiator primer (bearing the Cy5 fluorophore in 5′). Reactions were performed at 80° C. in the presence or in the absence of each substrate (lanes 1 to 8). Lane 9 shows a two steps reaction in which dNTPs were first incubated with either PolpP12_{Δ297-898} or PolpTN_{Δ231 923}, during 15 minutes, followed by the addition of the initiator primer. Left lane shows the MW ladder (SmartLadder 200 to 10000 bp (Eurogentec)).

FIG. 5A: merge of red and green channels;

FIG. 5B: green channel, Sybr green II fluorescence at 520 nm;

FIG. 5C: red channel, Cy5 fluorescence at 675 nm.

FIGS. 6A-C are a set of three photographs of a 1.5% agarose gel electrophoresis showing a template independent nucleic acid synthesis assay using PolpP12_{Δ297-898}, in comparison with the wild-type terminal deoxynucleotidyl transferase from calf thymus [TdT]. Reactions were performed at 37° C. or 70° C. using either a dGTP/dCTP mix or a dNTP mix as substrates. SF (dsDNA) Ladder: SmartLadder 100 to 1000 bp (Eurogentec); LF (dsDNA) Ladder: SmartLadder 200 to 10000 bp (Eurogentec).

FIG. 6A: merge of red and green channels;

FIG. 6B: red channel, Cy5 fluorescence at 675 nm;

FIG. 6C: green channel, Sybr green II fluorescence at 520 nm.

FIG. 7 is a photograph of a 15% urea-PAGE showing the incorporation of protected nucleoside triphosphates (3′-O-amino-dATP and 3′-O-azidomethyl-dATP), using PolpP12_{Δ297-898} at 60° C.

FIGS. 8A-C are a set of three figures showing the incorporation by PolpP12_{Δ297-898} of labeled nucleoside triphosphates with reversibly terminating aminoalkoxyl groups at 80° C.

FIG. 8A: 15% urea-PAGE showing the incorporation of 3′-O-amino dATP or 3′-O-amino dTTP at 80° C.;

FIG. 8B: analysis report of 3′-O-amino dATP incorporation at 80° C. Rf: relative migration distance;

FIG. 8C: analysis report of 3′-O-amino dTTP incorporation at 80° C. Rf: relative migration distance.

FIGS. 9A-C are a set of three figures showing the incorporation by PolpP12_{Δ297-898} of nucleoside triphosphates labeled with 3′-O-azidomethylene groups at 80° C.

FIG. 9A: 15% urea-PAGE showing the incorporation of 3′-O-azidomethyl dATP or 3′-O-azidomethyl dTTP at 80° C.;

FIG. 9B: analysis report of 3′-O-azidomethyl dATP incorporation at 80° C. R_{f}: relative migration distance;

FIG. 9C: analysis report of 3′-O-azidomethyl dTTP incorporation at 80° C. R_{f}: relative migration distance.

FIGS. 10A-B are a set of two figures showing the incorporation by PolpP12_{Δ297-898} of nucleoside triphosphates labeled with 3′-O-(N-methyl-anthraniloyl) groups at 70° C.

FIG. 10A: 15% urea-PAGE showing the incorporation of 3′-O-(N-methyl-anthraniloyl)-2′-dATP at 70° C.;

FIG. 10B: analysis report of 3′-O-(N-methyl-anthraniloyl)-2′-dATP incorporation at 70° C. Rf: relative migration distance.

FIGS. 11A-B are a set of two figures showing the incorporation by PolpP12_{Δ297-898} of nucleoside triphosphates labeled with 3′-O-(2-nitrobenzyl) groups at 70° C.

FIG. 11A: 15% urea-PAGE showing the incorporation of 3′-O-(2-nitrobenzyl)-2′-dATP at 70° C.;

FIG. 11B: analysis report of 3′-O-(2-nitrobenzyl)-2′-dATP incorporation at 70° C. Rf: relative migration distance.

FIG. 12 is a photograph of a 15% urea-PAGE showing the incorporation by PolpP12_{Δ297-898} at 80° C. of ribonucleoside triphosphates as well as deoxyuridine triphosphate.

FIGS. 13A-B are a set of two photographs of a 15% urea-PAGE showing the incorporation by PolpP12_{Δ297-898} of ribonucleoside triphosphates labeled with a 3′-O-propargyl group.

FIG. 13A: incorporation of 3′-O-propargyl GTP by PolpP12_{Δ297-898} at 70° C.;

FIG. 13B: negative control with calf thymus TdT at 37° C.

FIGS. 14A-D are a set of four figures photographs showing the incorporation by PolpP12_{Δ297-898} of deoxyinosine triphosphate and base-modified nucleosides triphosphate at 70° C.

FIG. 14A: structure of biotin-14-N6-(6-aminohexyl)-dATP;

FIG. 14B: structure of γ-[N-(biotin-6-amino-hexanoyl)]-(5-aminoallyl)-2′-dUTP;

FIG. 14C: structure of 5-(3-aminoallyl)-2′-dUTP;

FIG. 14D: 15% urea-PAGE showing the incorporation by PolpP12_{Δ297-898} of deoxyinosine, biotin-14-N⁶-(6-aminohexyl)-dATP, γ-[N-(biotin-6-amino-hexanoyl)]-(5-aminoallyl)-2′-dUTP or 5-(3-aminoallyl)-2′-dUTP at 70° C.

FIG. 15 is a photograph of an electrophoresis gel (SDS-PAGE) showing the purification of PolpTN2_{Δ311-923} and PolpTN2_{Δ90-96Δ311-923}. MW ladder: molecular weight ladder.

FIG. 16 is a photograph of a 1.5% agarose gel electrophoresis showing a template-independent nucleic acid synthesis assay using PolpTN2_{Δ311-923} and PolpTN2_{Δ90-96Δ311-923}, and carried out in the presence or in the absence of dNTPs and/or the initiator sequence (bearing the Cy5 fluorophore in 5′). Reactions were performed at 70° C. in the presence or in the absence of each substrate. MW ladder is SmartLadder 200 to 10000 bp (Eurogentec). Red channel, Cy5 fluorescence at 675 nm.

### EXAMPLES

The present invention is further illustrated by the following examples.

### Example 1

Phylogenetic Analysis of Archaeal DNA Primases

A phylogenetic analysis was performed to highlight the evolutionary relationship between ten selected archaeal DNA primases:
a functionally active fragment of the Pyrococcus sp. 12-1 DNA primase (PolnP12_{Δ297-898}) having the amino acid sequence of SEQ ID NO: 2;
a functionally active fragment of the Thermococcus sp. CIR10 DNA primase (PolpTCIR10_{Δ303-928}) having the amino acid sequence of SEQ ID NO: 5;
a functionally active fragment of the Thermococcus peptonophilus DNA primase (PolpTpep_{Δ295-914}) having the amino acid sequence of SEQ ID NO: 7;
a functionally active fragment of the Thermococcus celericrescens DNA primase (PolpTcele_{Δ295-913}) having the amino acid sequence of SEQ ID NO: 9;
a functionally active fragment of the Thermococcus nautili sp. 30-1 DNA primase (PolpTN2_{Δ311-923}) having the amino acid sequence of SEQ ID NO: 11.
Pyrococcus furiosus DNA primase having the amino acid sequence of SEQ ID NO: 12;
Thermococcus kodakarensis DNA primase having the amino acid sequence of SEQ ID NO: 13;
Saccharolobus solfataricus DNA primase having the amino acid sequence of SEQ ID NO: 14;
Pyrococcus horikoshii DNA primase having the amino acid sequence of SEQ ID NO: 15; and
Archaeoglobus fulgidus DNA primase having the amino acid sequence of SEQ ID NO: 16.

SEQ ID NO: 12 represents the amino acid sequence of the protein “DNA primase catalytic subunit PriS” from Pyrococcus furiosus with NCBI Reference Sequence WP_011011222 version 1 of 2019-06-20.

SEQ ID NO: 12
MLMREVTKEERSEFYSKEWSAKKIPKFIVDTLESREFGFDHNGEGPSDR
KNQYSDIRDLEDYIRATSPYAVYSSVAFYENPREMEGWRGAELVFDIDA
KDLPLKRCNHEPGTVCPICLEDAKELAKDTLIILREELGFENIHVVYSG
RGYHIRILDEWALQLDSKSRERILAFISASEIENVEEFRRFLLEKRGWF
VLKHGYPRVFRLRLGYFILRVNVPHLLSIGIRRNIAKKILDHKEEIYEG
FVRKAILASFPEGVGIESMAKLFALSTRFSKAYFDGRVTVDIKRILRLP
STLHSKVGLIATYVGTKEREVMKFNPFRHAVPKFRKKEVREAYKLWRES
LEYE

SEQ ID NO: 13 represents the amino acid sequence of the protein “DNA primase catalytic subunit PriS” from Thermococcus kodakarensis with NCBI Reference Sequence WP 011250742 version 1 of 2019-06-15.

SEQ ID NO: 13
MSKLLREVTPEERRLYYSGEWDAKKLPEFIVESIERREFGFDHTGEGPS
DRKNAFSDVRDLEDYIRATAPYAAYSSVAFYRNPQEMEGWLGAELVFDI
DAKDLPLRRCQNEHPSGQVCPICLEDAKELARDTLIILKEDFGFENIHV
VYSGRGYHIRVIDEWALKLDSKARERILSYVSAAEEVTFDDIQKRYIML
SSGYFRVFRLRFGYFIQRINENHLKNIGLKRSTAEKLLDEKTRQDIVEK
FVNKGLLAAFPEGVGYRTLLRLFGLSTTFSKAYFDGRVTVDLKRILRLP
STLHSKVGLVATYIGSDEKRLEKFDPFKDAVPEFRKEEVQKAYQEWKEL
HEG

SEQ ID NO: 14 represents the amino acid sequence of the protein “DNA primase small subunit PriS” from Saccharolobus solfataricus with NCBI Reference Sequence WP_009989180 version 1 of 2021-03-14.

SEQ ID NO: 14
MGTFTLHQGQTNLIKSFFRNYYLNAELELPKDMELREFALQPFGSDTYV
RHLSFSSSEELRDYLVNRNLPLHLFYSSARYQLPSARNMEEKAWMGSDL
LFDIDADHLCKLRSIRFCPVCGNAVVSEKCERDNVETLEYVEMTSECIK
RGLEQTRNLVEILEDDFGLKPKVYFSGNRGFHVQVDCYGNCALLDSDER
KEIAEYVMGIGVPGYPGGSENAPGWVGRKNRGINGVTIDEQVTIDVKRL
IRIPNSLHGKSGLIVKRVPNLDDFEFNETLSPFTGYTIFLPYITIETEV
LGSIIKLNRGIPIKIKSSIGIYLHLRNLGEVKAYVR

SEQ ID NO: 15 represents the amino acid sequence of the protein “DNA primase small subunit PriS” from Pyrococcus horikoshii with NCBI Reference Sequence WP_010884304 version 1 of 2019-06-20.

SEQ ID NO: 15
MLLREVTREERKNFYTNEWKVKDIPDFIVKTLELREFGFDHSGEGPSDR
KNQYTDIRDLEDYIRATAPYAVYSSVALYEKPQEMEGWLGTELVFDIDA
KDLPLRRCEHEPGTVCPICLNDAKEIVRDTVIILREELGFNDIHIIYSG
RGYHIRVLDEWALKLDSKSRERILSFVSASEIEDVEEFRKLLLNKRGWF
VLNHGYPRAFRLRFGYFILRIKLPHLINAGIRKSIAKSILKSKEEIYEE
FVRKAILAAFPQGVGIESLAKLFALSTRFSKSYFDGRVTVDLKRILRLP
STLHSKVGLIAKYVGTNERDVMRFNPFKHAVPKFRKEEVKVEYKKFLES
LGT

SEQ ID NO: 16 represents the amino acid sequence of the protein “DNA primase small subunit PriS” from Archaeoglobus fulgidus with NCBI Reference Sequence WP_048064280 version 1 of 2019-06-15.

SEQ ID NO: 16
MLTKLFLKKKFEEYYSKNEVELPRKFKNREFAFVPLELLPDFVMHRHIS
FRSETDFRAYILSNVPAHIYFSSAYYERPAEDKMENKGWLGADLIFDID
ADHLPVKAQSFEKALEMAKREIKKLTAVLRADFGIRDMKIYFSGGRGYH
VHVHDEEFLSLGSAERREIVDYLRLNSPKIVVEDRFANSNAAKRVLNYL
RKKLEEDERLTSKLKIKPADLKKEKLTKKVIRAVEKFDYSALSIYIDAP
VTADVKRLIRLPGSLHGKTGLRVTEVEDIESFNPLKDALAFGDEAVVVK
VARKLNLSIGDFSGKIYPGRVKLPEYAAVFLICRGDASYDS

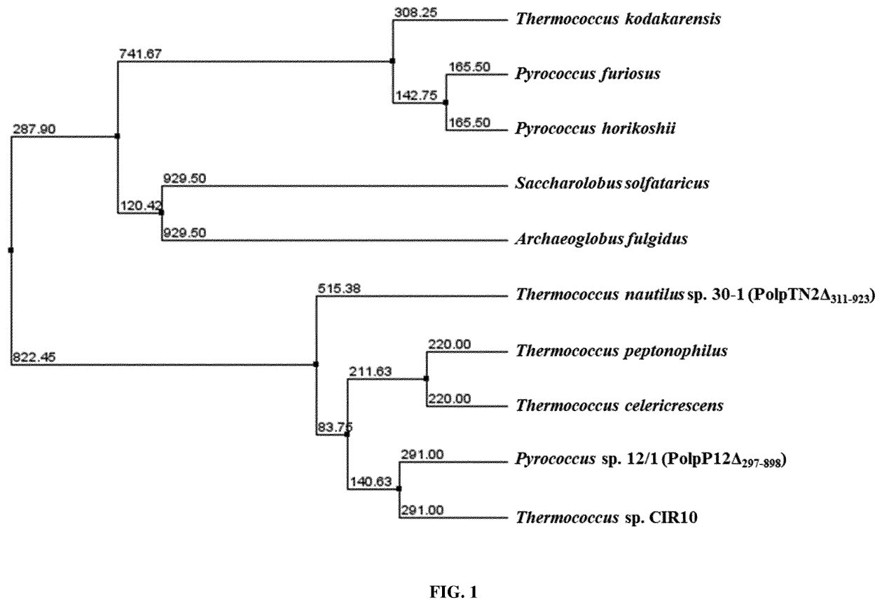

As seen on the phylogenetic tree (FIG. 1—generated using BLOSUM62 with average distance), we can observe a high degree of evolutionary divergence between the four DNA primases from Pyrococcus sp. 12-1, Thermococcus sp. CIR10, Thermococcus peptonophilus and Thermococcus celericrescens on one hand, and the DNA primases from Pyrococcus furiosus, Thermococcus kodakarensis, Saccharolobus solfataricus, Pyrococcus horikoshii, Archaeoglobus fulgidus and Thermococcus nautili sp. 30-1 on the other hand.

This evolutionary divergence is strengthened by the identity matrix (Table 1), which shows a very low identity score between the first four DNA primases and the later six DNA primases (maximum 11.1%).

Although the first four DNA primases show a higher degree of identity with the DNA primase from Thermococcus nautili sp. 30-1, both the phylogenetic tree (FIG. 1) and the

TABLE 1

| | P. | T. | S. | P. | A. | P. sp. | T. nautili | T. sp. | T. | T. |
|---|---|---|---|---|---|---|---|---|---|---|
| DNA primase | furiosus | kodakarensis | solfataricus | horikoshii | fulgidus | 12-1 | sp. 30-1 | CIR10 | peptonophilus | celericrescens |
| P. | ID | 0.662 | 0.12 | 0.786 | 0.185 | 0.074 | 0.093 | 0.088 | 0.111 | 0.106 |
| furiosus | | | | | | | | | | |
| T. | 0.662 | ID | 0.129 | 0.639 | 0.187 | 0.071 | 0.068 | 0.077 | 0.1 | 0.091 |
| kodakarensis | | | | | | | | | | |
| S. | 0.12 | 0.129 | ID | 0.14 | 0.221 | 0.077 | 0.065 | 0.08 | 0.063 | 0.081 |
| solfataricus | | | | | | | | | | |
| P. | 0.786 | 0.639 | 0.14 | ID | 0.196 | 0.08 | 0.082 | 0.085 | 0.1 | 0.1 |
| horikoshii | | | | | | | | | | |
| A. | 0.185 | 0.187 | 0.221 | 0.196 | ID | 0.052 | 0.049 | 0.063 | 0.055 | 0.046 |
| fulgidus | | | | | | | | | | |
| P. | 0.074 | 0.071 | 0.077 | 0.08 | 0.052 | ID | 0.511 | 0.739 | 0.614 | 0.614 |
| sp. 12-1 | | | | | | | | | | |
| T. nautili | 0.093 | 0.068 | 0.065 | 0.082 | 0.049 | 0.511 | ID | 0.523 | 0.503 | 0.515 |
| sp. 30-1 | | | | | | | | | | |
| T. sp. | 0.088 | 0.077 | 0.08 | 0.085 | 0.063 | 0.739 | 0.523 | ID | 0.588 | 0.611 |
| CIR10 | | | | | | | | | | |
| T. | 0.111 | 0.1 | 0.063 | 0.1 | 0.055 | 0.614 | 0.503 | 0.588 | ID | 0.871 |
| peptonophilus | | | | | | | | | | |
| T. | 0.106 | 0.091 | 0.081 | 0.1 | 0.046 | 0.614 | 0.515 | 0.611 | 0.871 | ID |
| celericrescens | | | | | | | | | | |

identity matrix (Table 1) still demonstrate their distant relationship. Indeed, from an evolutionary point of view, the DNA primase from Thermococcus nautili sp. 30-1 appears to be out of the group with a maximal identity score of 52.3%.

### Example 2

PolpP12_{Δ297-898} has a Template-Independent Terminal Nucleotidyl Transferase Activity and is Devoid of an Ab-Initio Single-Stranded Nucleic Acid Synthesis Activity

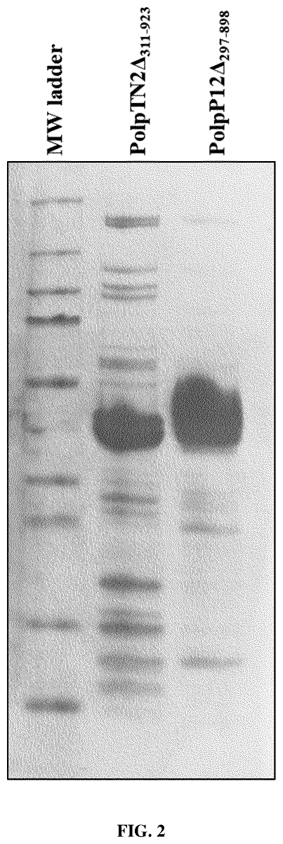

The N-terminal domain of the DNA primase from Pyrococcus sp. 12-1 (PolpP12_{Δ297-898} having the amino acid sequence of SEQ ID NO: 2) and from Thermococcus nautili sp. 30-1 (PolpTN2_{Δ311-923} having the amino acid sequence of SEQ ID NO: 11) were expressed and purified following a protocol adapted from WO2011098588 and Gill et al., 2014 (Nucleic Acids Res. 42(6):3707-3719) (FIG. 2).

A template-independent nucleic acid synthesis assay was carried out with either PolpP12_{Δ297-898} or PolpTN2_{Δ311-923}, at 60° C., 70° C. and 80° C., using a single stranded nucleic acid primer as initiator sequence (bearing a Cy5 fluorophore in 5′).

Three different conditions were tested:
a: initiator sequence only; no enzyme, no dNTP;
b: initiator sequence+enzyme; no dNTP;
c: initiator sequence+enzyme+dNTP mix (unprotected).

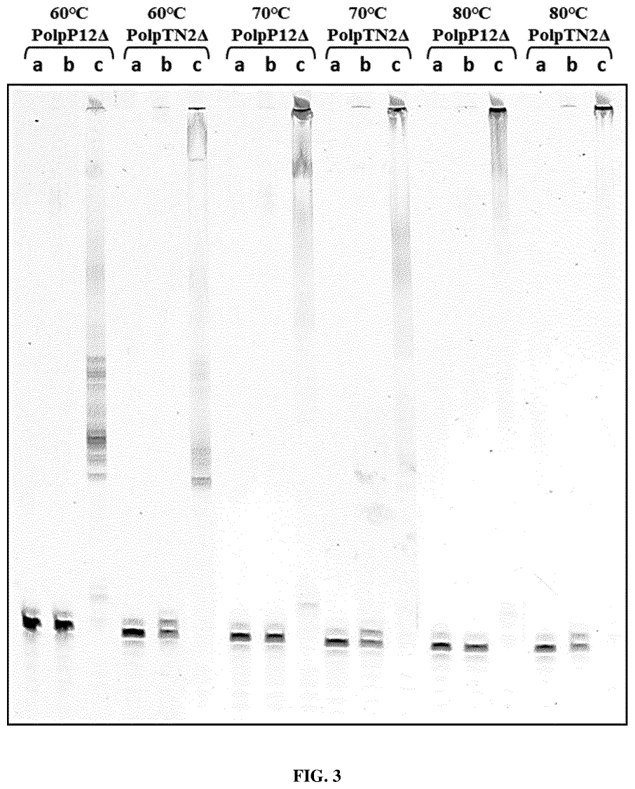

As seen on FIG. 3, both PolpP12_{A}297-898 and PolpTN2_{Δ311-923} exhibit an untemplated terminal nucleotidyl transferase activity for each tested temperature, when using a mixture of all four dNTPs as substrate. However, it is worth noting that at 70° C. and 80° C., most of the newly synthetized nucleic acids are several hundred bases long, hence cannot be resolved on a 15% urea-PAGE gel and remain in the well.

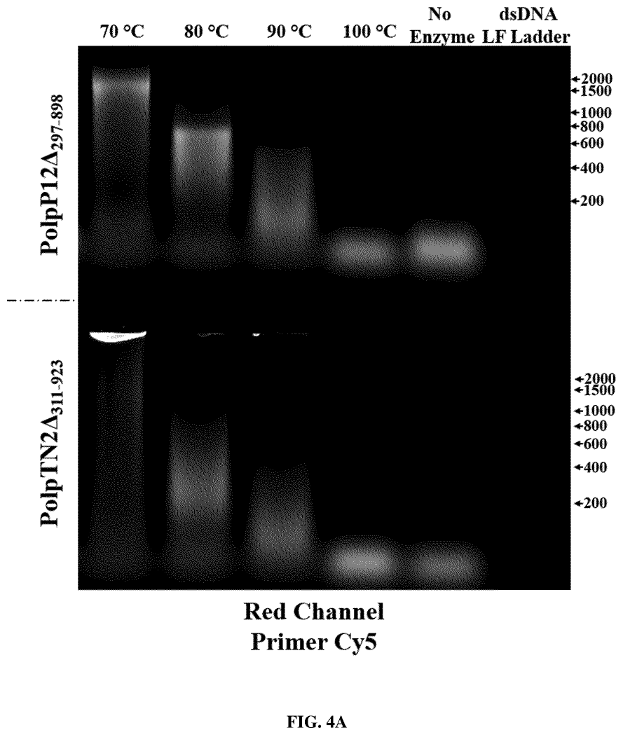

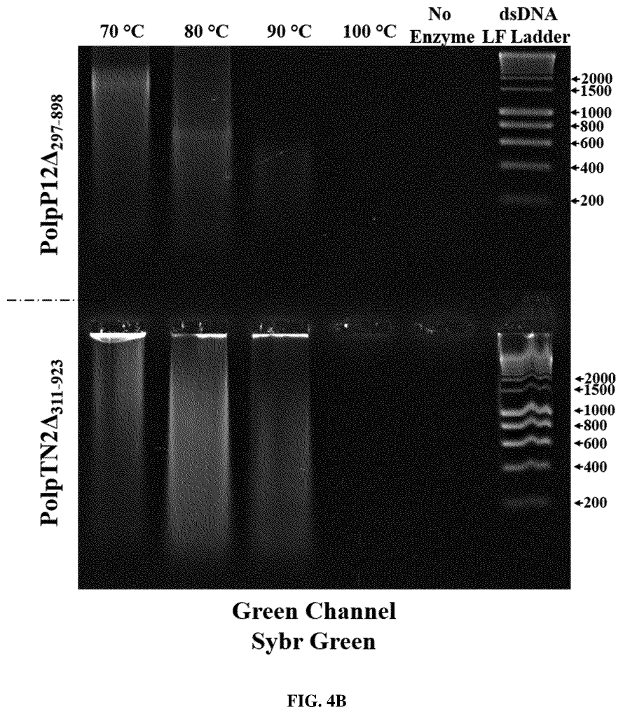

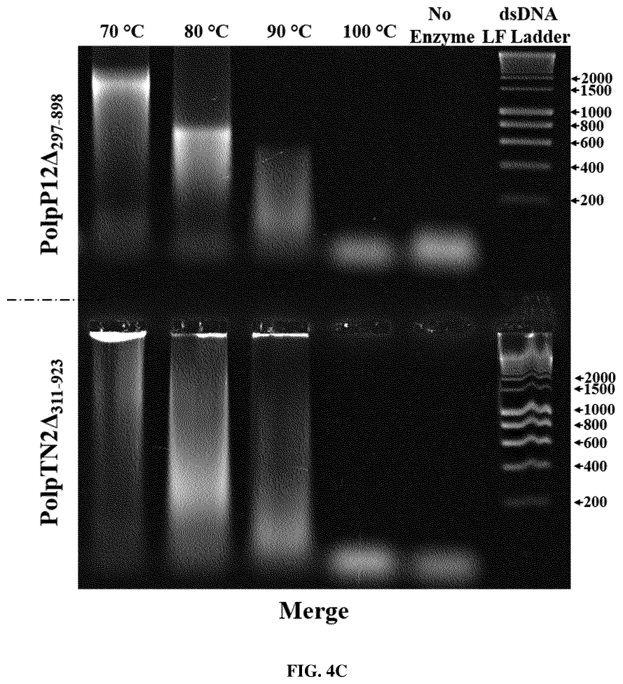

Thus, to analyze the effect of high temperatures on PolpP12_{Δ297-898} and PolpTN2_{Δ311-923} activities, a template-independent nucleic acid synthesis assay was performed as previously described, at 70° C., 80° C., 90° C. or 100° C. and resolved by agarose gel electrophoresis (FIG. 4). The terminal transferase activity was specifically evaluated by following the polymerization of the fluorescent primer (bearing a Cy5 fluorophore in 5′), recorded at 675 nm (red channel). Total nucleic acid synthesis and molecular weight markers were stained using Sybr Green II and recorded at 520 nm (green channel).

As shown on FIGS. 4A and C, both enzymes exhibit a strong template-independent terminal nucleotidyl transferase activity, which is demonstrated by the polymerization of the Cy5-labeled initiator primer (FIG. 4A). These activities reach a maximum of polymerization at 70° C. and gradually decrease upon increasing temperatures, up to 100° C. However, in contrast to PolpP12_{Δ297-898}, PolpTN2_{Δ311-923} exhibits a diffuse migration pattern at 70° C., 80° C. and 90° C., when stained with Sybr Green II (FIG. 4B), which does not colocalize with the Cy5-labeled initiator primer (Cf. FIGS. 4A and C). Although this intriguing result may arise from a migration issue, another explanation would be the presence of an unexpected competing activity, such as an ab-initio single-stranded nucleic acid synthesis activity.

Interestingly, Béguin et al. have demonstrated that a combination of the full length PolpTN2 primase and the PolB DNA polymerase in presence of deoxyribonucleoside triphosphates leads to the ab-initio synthesis of long double stranded DNA fragments (i.e., without template DNA nor oligonucleotide primer). However, this phenomenon requires the presence of both enzymes and is not observed when only PolpTN2 is reacted with a dNTP mix (Béguin et al., 2015. Extremophiles. 19(1):69-76). In contrast, our results suggest that PolpTN2_{Δ311-923} alone might be able to synthesis long fragments of single stranded nucleic acids de novo.

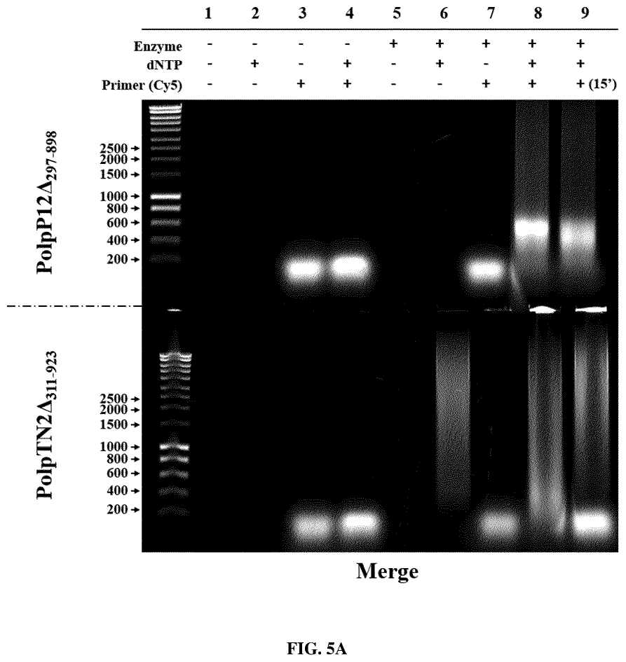

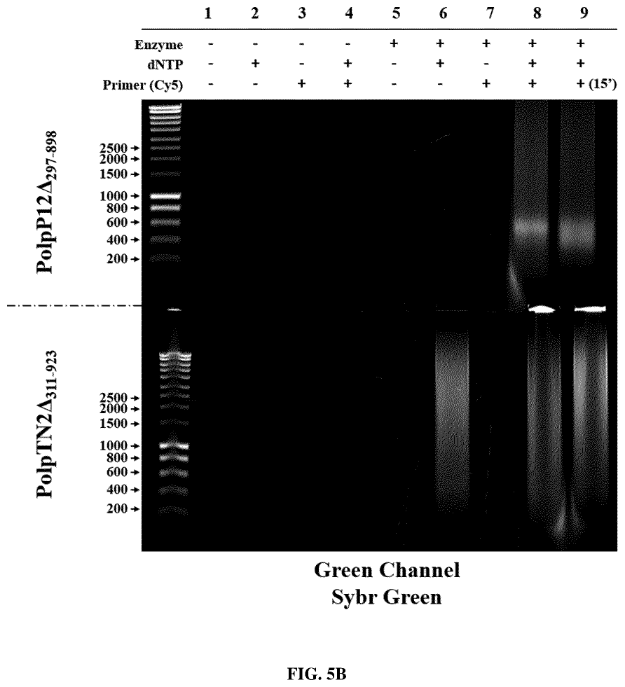

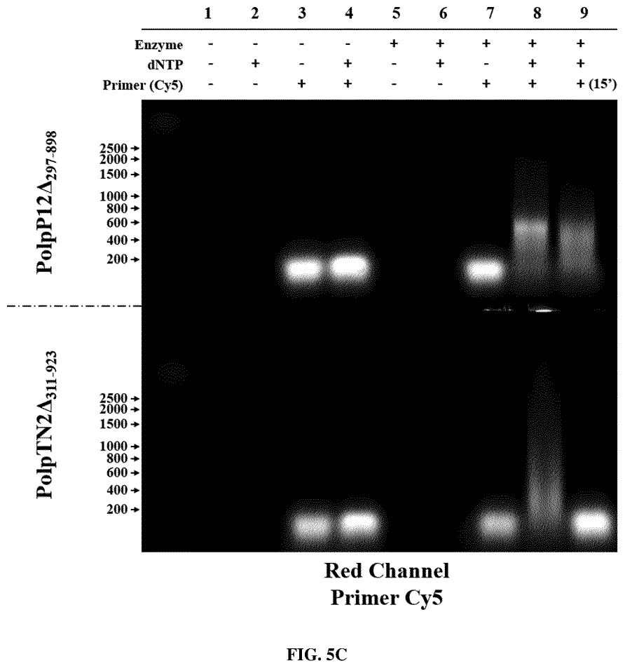

To further investigate this phenomenon, both PolpP12_{Δ297-898} and PolpTN2_{Δ311-923} were subjected to a template-independent nucleic acid synthesis assay (FIG. 5), carried out in the presence or in the absence of dNTPs and/or the initiator primer (bearing the Cy5 fluorophore in 5′). The terminal transferase activity was specifically evaluated by following the polymerization of the fluorescent primer, recorded at 675 nm (red channel). Total nucleic acid synthesis and molecular weight markers were stained using Sybr Green II and recorded at 520 nm (green channel).

Nine different conditions were tested:
1: no enzyme, no initiator sequence, no dNTP;
2: dNTP only; no enzyme, no initiator sequence;
3: initiator sequence only; no enzyme, no dNTP;
4: initiator sequence+dNTP mix; no enzyme;
5: enzyme only, no initiator sequence, no dNTP;
6: enzyme+dNTP mix; no initiator sequence;
7: enzyme+initiator sequence; no dNTP;
8: enzyme+dNTP mix+initiator sequence;
9: enzyme+dNTP mix+initiator sequence (added after 15 minutes incubation); As shown on FIG. 5, neither PolpP12_{Δ297-898} nor PolpTN2_{Δ311-923} is able to synthesize nucleic acids in the absence of dNTPs (FIG. 5; lanes 1, 3, 5 and 7) while the association of dNTPs with the initiator primer leads to the synthesis of long nucleic acids fragments (FIG. 5A; lanes 8 and 9). Interestingly, in the absence of the initiator primer (FIG. 5; lanes 1, 2, 5 and 6) PolpTN2_{Δ311-923} readily exhibits a strong polymerase activity, upon the addition of dNTPs (FIGS. 5A and 5B; lane 6). Separated channel analysis reveals that this activity is independent of an initiator primer, as demonstrated by the absence of Cy5 fluorescence (FIG. 5C; lane 6), thus confirming the ab-initio single-stranded nucleic acid synthesis activity of PolpTN2_{Δ311-923}. Conversely, in the same experimental conditions, PolpP12_{Δ297-898} does not synthesize nucleic acids, as demonstrated by a total absence of fluorescence in both channels (FIGS. 5A, 5B and 5C; lane 6), indicating that this enzyme is devoid of an ab-initio single-stranded nucleic acid synthesis activity.

To further investigate the impact of such ab-initio single-stranded nucleic acid synthesis activity on the ability of PolpP12_{Δ297-898} and PolpTN2_{Δ311-923} to extend a single stranded nucleic acid fragment, a competition assay was conducted by separating both reactions (FIG. 5, lane 9). To realize this experiment, both enzymes were first incubated with dNTPs for 15 minutes, to perform a template-independent primase reaction, before adding the initiator primer for another 15 minutes of incubation, to perform a template-independent primer extension reaction. As expected, PolpP12_{Δ297-898} was found to extend the initiator primer (FIG. 5C, lane 9) as previously described, indicating that a pre-incubation with a dNTP mix does not affect its terminal nucleotidyl transferase activity. In contrast, a strong diffuse migration pattern could be observed for PolpTN2_{Δ311-923}, after Sybr Green II staining (FIG. 5B, lane 9), while the initiator primer was found to migrate up to the dye front (FIG. 5C, lane 9), similarly to the negative control (FIG. 5C, lane 3). Thus, this result indicates that PolpTN2_{Δ311-923} is not able to extent the initiator primer in these experimental conditions.

Therefore, these results demonstrate the negative side effect of the ab-initio single-stranded nucleic acid synthesis activity of PolpTN2_{Δ311-923} on its ability to perform a template-independent terminal nucleotidyl transferase reaction, for which a strong competition can be observed. Conversely, PolpP12_{Δ297-898} appears devoid of ab-initio single-stranded nucleic acid synthesis activity, and rather acts as a true terminal nucleotidyl transferase, capable of extending an initiator primer to create long nucleic acids fragments, strengthening its application for industrial nucleic acids synthesis.

### Example 3

PolpP12_{Δ297-898} has a Higher Processivity than a Member of X-Family Polymerases

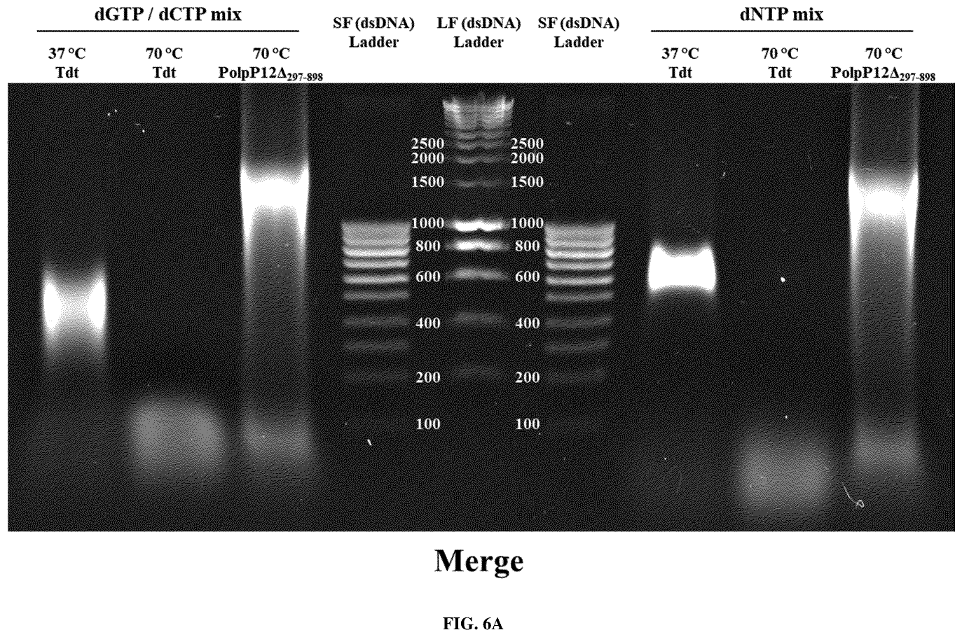

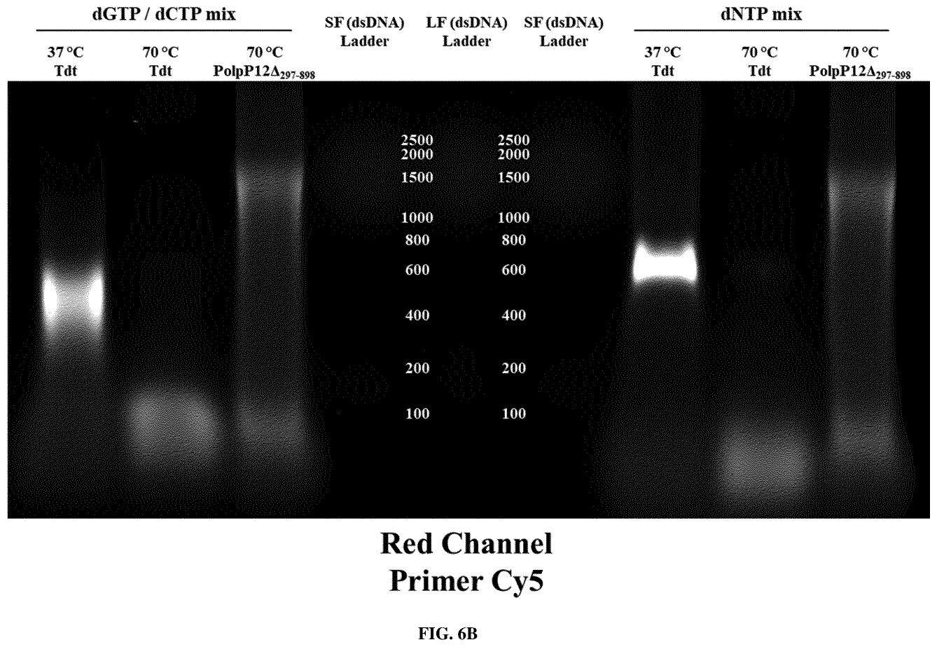

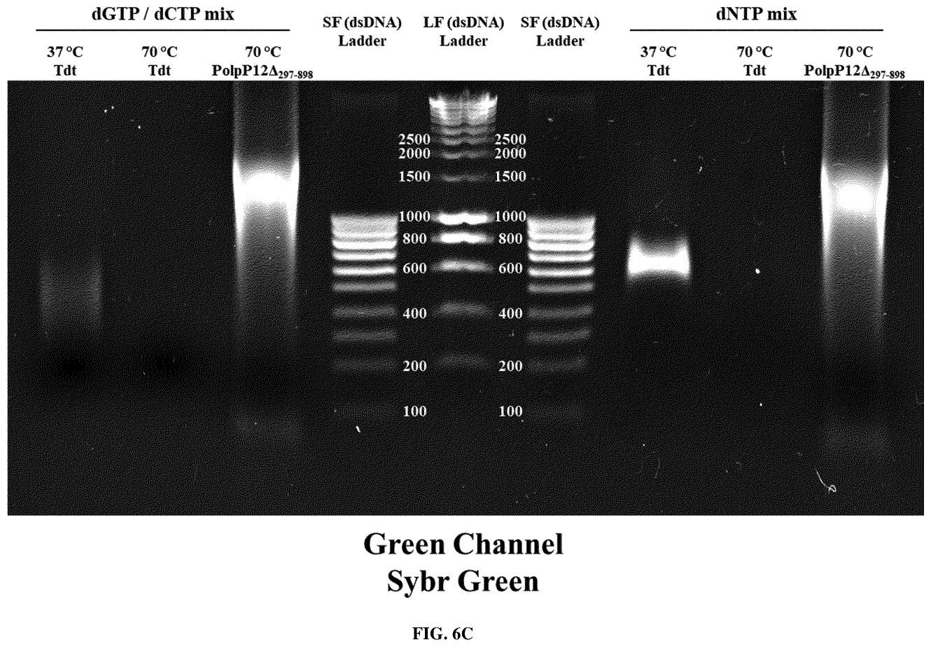

To test whether the use of PolpP12_{Δ297-898} presents an industrial advantage over the use of X-family polymerases, a terminal transferase activity assay was carried out at 70° C., and was compared to that of the recombinant terminal deoxynucleotidyl transferase (TdT) from calf thymus at either 37° C. or 70° C. (FIG. 6).

Experiments were performed using a single-stranded nucleic acid primer as initiator sequence (bearing a Cy5 fluorophore in 5′) and in the presence of a dCTP/dGTP mix or a mixture of all four dNTPs as substrates. The terminal transferase activity was specifically evaluated by following the polymerization of the fluorescent primer, recorded at 675 nm (red channel). Total nucleic acid synthesis and molecular weight markers were stained using Sybr Green II and recorded at 520 nm (green channel). TdT was obtained from New England Biolabs (M0315S).

As seen on FIGS. 6A-C, the transferase activity of PolpP12_{Δ297-898} at 70° C. results in the synthesis of long nucleic acid fragments (1.5 kb), either with a dCTP/dGTP mix or with a dNTP mix, when compared to TdT that synthetize only short fragments at 37° C. (400 kb). In addition, no elongation products are observed for TdT at 70° C., neither with a dCTP/dGTP mix nor with a dNTP mix, demonstrating its lack of thermostability.

Therefore, the industrial use of PolpP12_{Δ297-898} appears more promising than the use of TdT for the synthesis of long nucleic acids. This is especially true for the synthesis of GC-rich sequences, such as the one found in microsatellites, which tend to create highly stable secondary structures and that require temperatures higher than 60° C. to break their hydrogen bonding network.

### Example 4

PolpP12_{Δ297-898} is Capable of Incorporating Protected Nucleosides Triphosphate

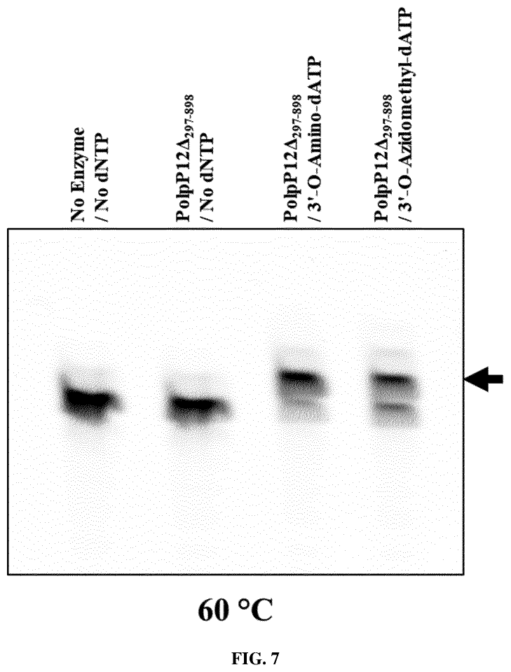

A terminal transferase activity assay was carried out with PolpP12_{Δ297-898} at 60° C. (FIG. 7) using 3′-O-amino dATP or 3′-O-azidomethyl dATP and a single stranded nucleic acid primer as initiator sequence (bearing a Cy5 fluorophore in 5′).

Four different conditions were tested:
a: initiator sequence only; no enzyme, no dNTP at 60° C.;
b: initiator sequence+enzyme; no dNTP at 60° C.;
c: initiator sequence+enzyme+3′-O-amino dATP at 60° C.;
d: initiator sequence+enzyme+3′-O-azidomethyl dATP at 60° C.;

Thus, PolpP12_{Δ297-898} was found to naturally incorporate 3′-reversible terminating nucleotides at 60° C., as demonstrated by the higher migration pattern of the initiator primer, when compared to the negative control (FIG. 7). Such a surprising behavior is a very uncommon feature among polymerases, as most of them usually require site directed mutagenesis of their catalytic pocket to relieve the steric hindrance that block the incorporation of 3′-reversible terminating nucleotides.

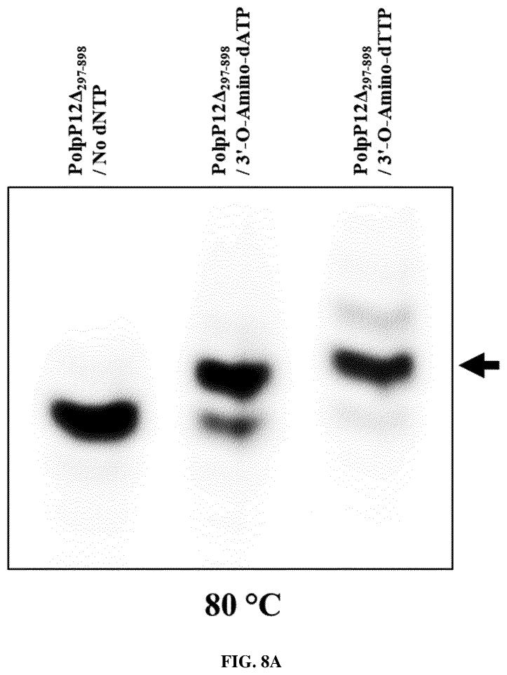

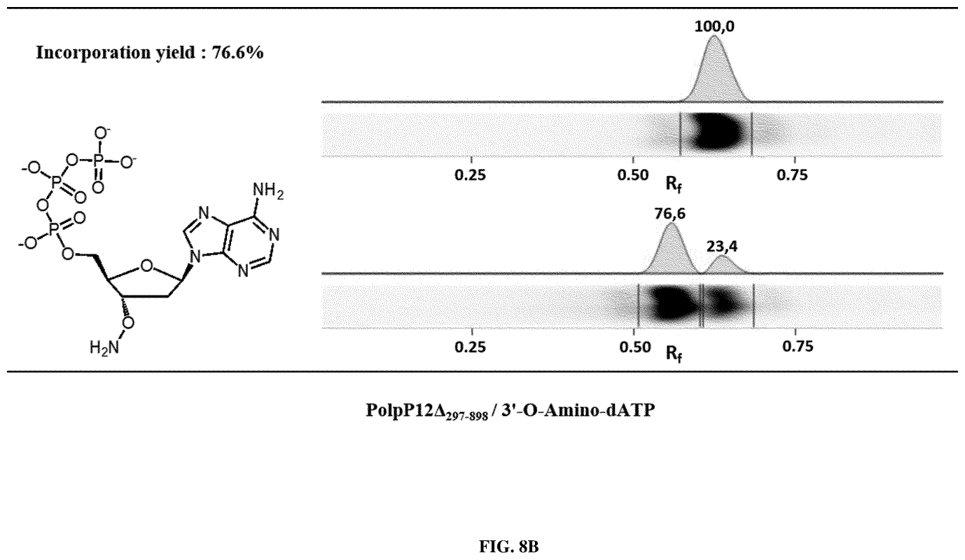

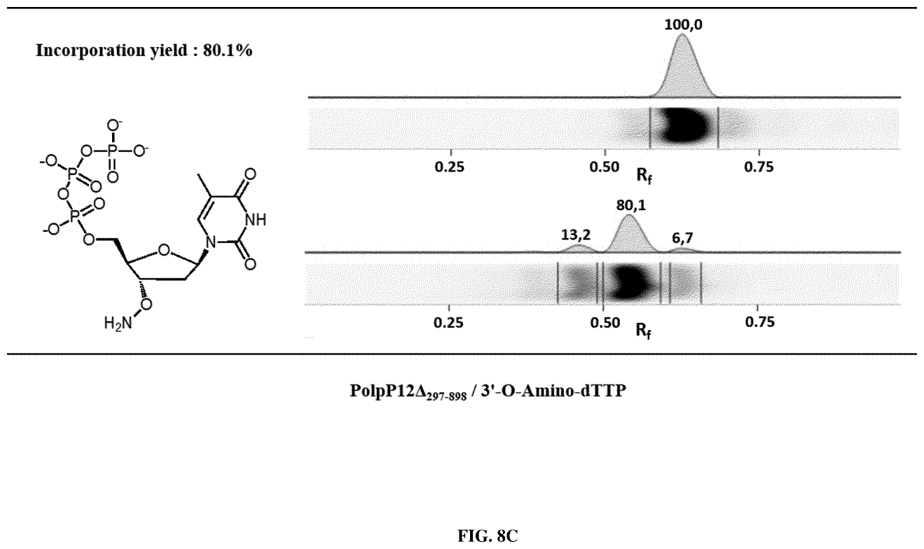

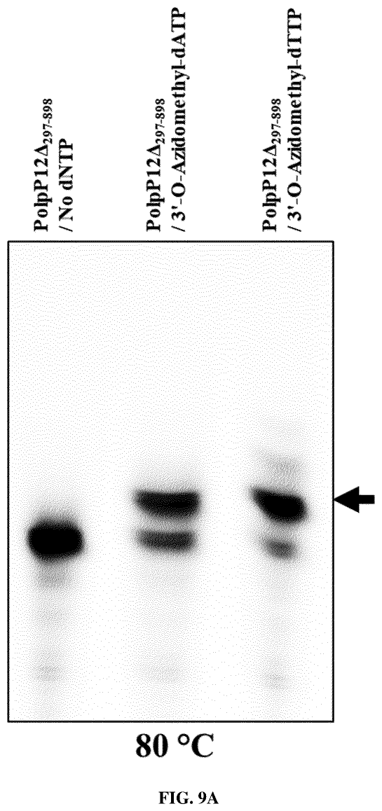

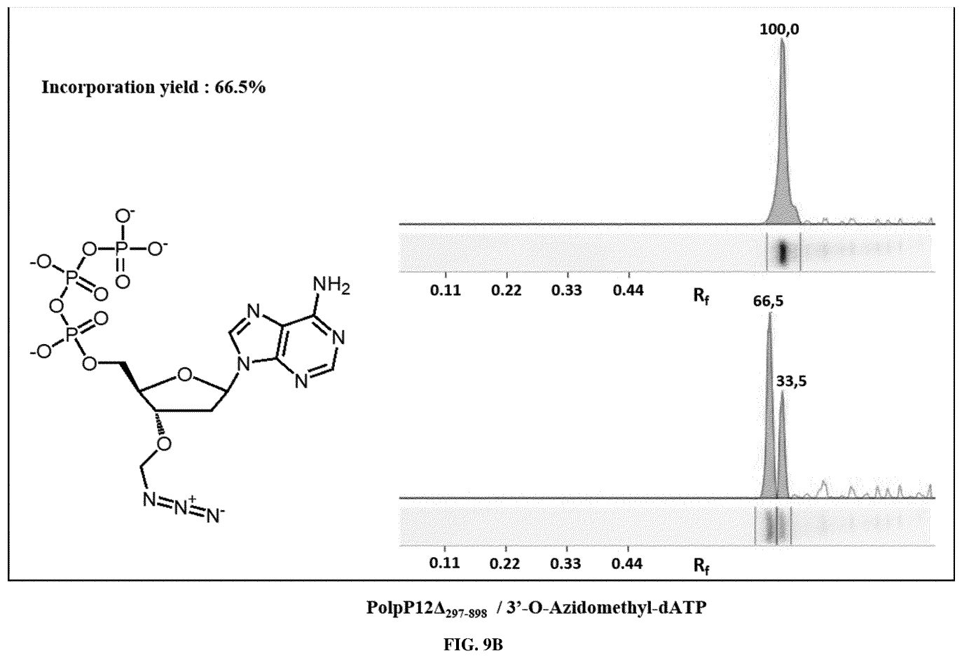

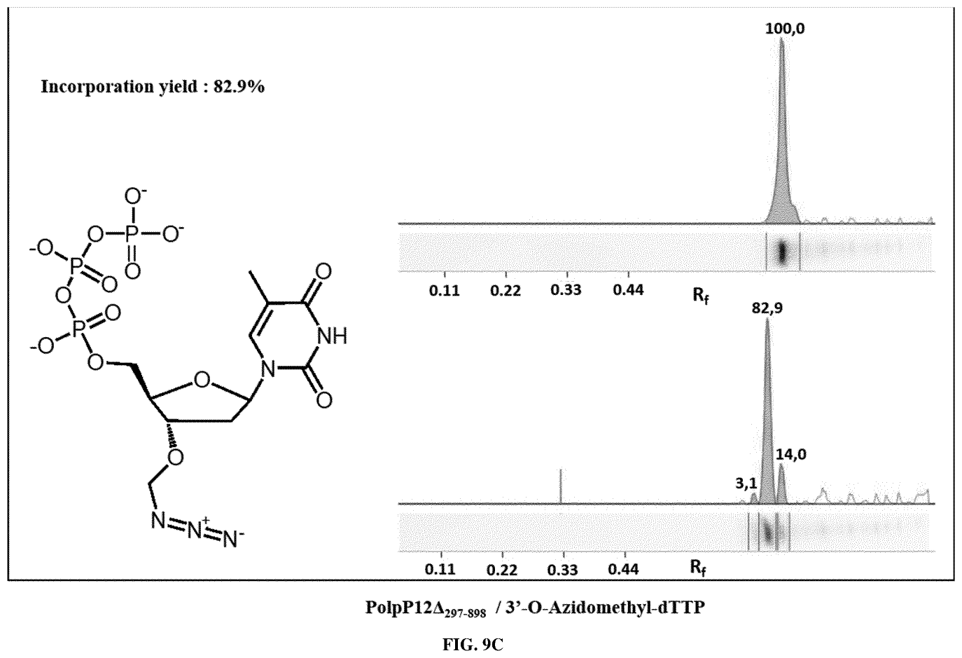

To further investigate the effect of higher temperatures on the ability of PolpP12_{Δ297-898} to incorporate 3′-reversible terminating nucleotides, a terminal transferase activity assay was carried out at 80° C. using 3′-O-amino dNTPs (FIG. 8) or 3′-O-azidomethyl dNTP (FIG. 9) and a single stranded nucleic acid primer as initiator sequence (bearing a Cy5 fluorophore in 5′).

Three different conditions were tested in each case:
initiator sequence+enzyme; no dNTP at 80° C.;
initiator sequence+enzyme+3′-O-amino-dATP or 3′-O-azidomethyl dATP at 80° C.;
initiator sequence+enzyme+3′-O-amino dTTP or 3′-O-azidomethyl dTTP at 80° C.

As previously shown, PolpP12_{Δ297-898} was found to efficiently incorporate 3′-reversible terminating nucleotides at 80° C., as demonstrated by the higher migration pattern of the initiator primer, when compared to negative controls (FIGS. 8A and 9A).

Furthermore, it was found to incorporate both purine-type and pyrimidine-type nucleobases, with a yield of 76.6% and 80.1% for 3′-O-amino dATP and 3′-O-amino dTTP respectively (FIGS. 8B and C), while the incorporation of 3′-O-azidomethyl dATP and 3′-O-azidomethyl dTTP led to yields of 66.5% and 82.9%, respectively (FIGS. 9B and C).

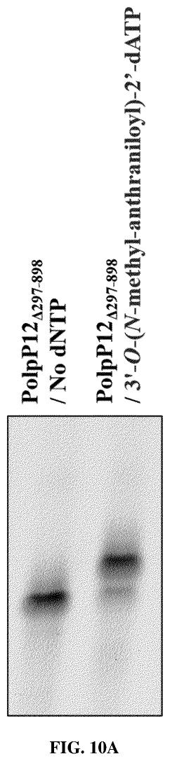

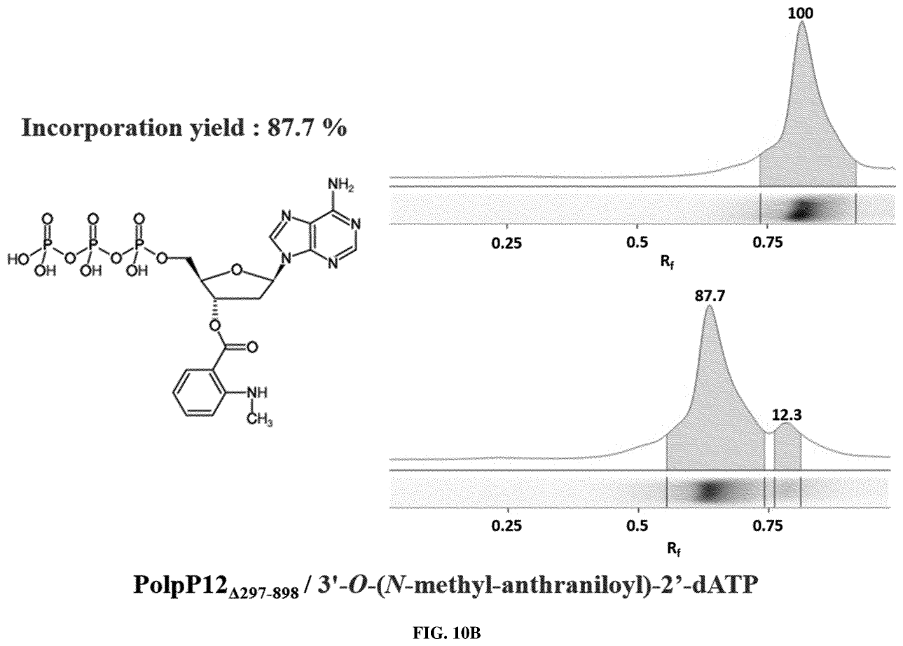

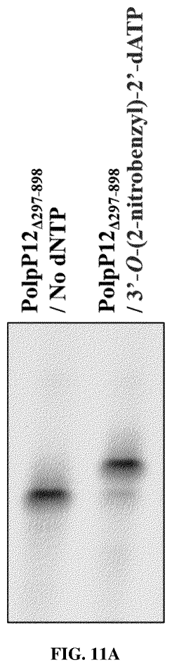

A terminal transferase activity assay was then carried out with PolpP12_{Δ297-898} at 70° C. using 3′-reversible terminating nucleotides bearing larger protecting groups, namely 3′-0-(N-methyl-anthraniloyl)-2′-dATP (FIG. 10) or 3′-O-(2-nitrobenzyl)-2′-dATP (FIG. 11), in presence of a single stranded nucleic acid primer as initiator sequence (bearing a Cy5 fluorophore in 5′).

For each experiment, two different conditions were tested:
initiator sequence+enzyme; no dNTP at 70° C.;
initiator sequence+enzyme+3′-O-(N-methyl-anthraniloyl)-2′-dATP (FIG. 10) or 3′-O-(2-nitrobenzyl)-2′-dATP (FIG. 11) at 70° C.

PolpP12_{Δ297-898} was found to incorporate nucleotides bearing large terminating groups on their 3′ position, as demonstrated by the higher migration pattern of the initiator primer, when compared to the negative control (FIGS. 10 and 11). In addition, these incorporations appear very efficient as judged by the high yields obtained, reaching up to 87.7% and 82% for 3′-O-(N-methyl-anthraniloyl)-2′-dATP and 3′-O-(2-nitrobenzyl)-2′-dATP, respectively.

Therefore, incorporation of such large functional groups on the 3′ position of nucleotides indicates that steric hinderance is neither a limitation nor a critical parameter for PolpP12_{Δ297-898} activity, and further allows a wide range of modifications with a broad spectrum of applications. Indeed, 3′-O-(N-methyl-anthraniloyl)-2′-dATP, also known as MANT-dATP, is a fluorescent nucleotide (κ_{exc} 355 nm/λₑₘ 448 nm) which can be used as a quantitative reporter during the nucleic acid synthesis process. Similarly, 3′-O-(2-nitrobenzyl)-2′-dATP exhibits an attractive industrial feature that arise from its photolabile protecting group, indicating that PolpP12_{Δ297-898} can be used in a process involving a photo-deprotection step instead of chemical deprotection.

### Example 5

PolpP12_{Δ297-898} is Capable of Incorporating Ribonucleosides Triphosphate and Deoxyuridine Triphosphate

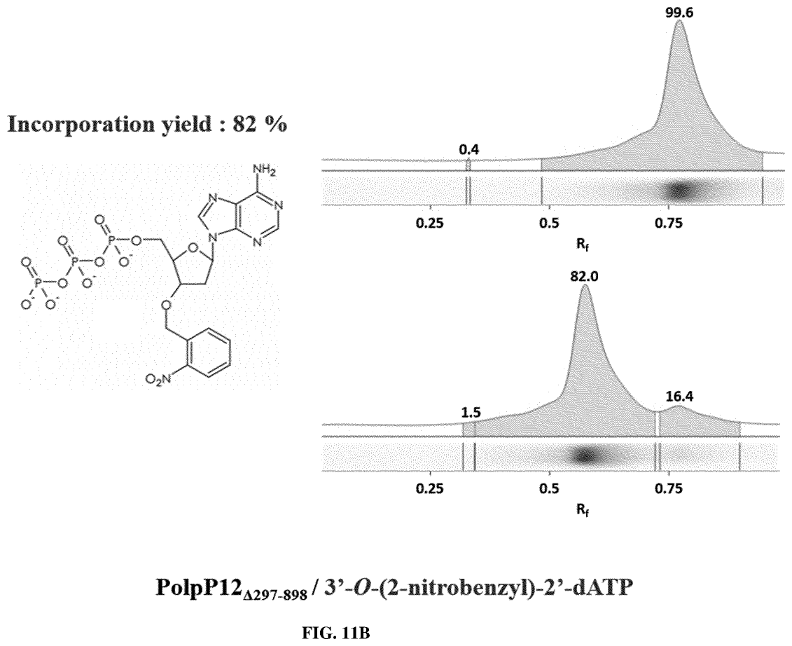

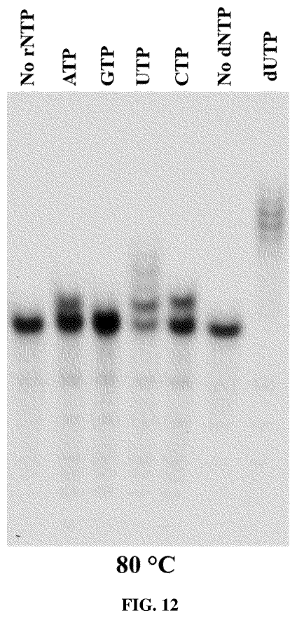

A terminal transferase activity assay was carried out with PolpP12_{Δ297-898} at 80° C. (FIG. 12) using rNTP or dUTP and a single stranded nucleic acid primer as initiator sequence (bearing a Cy5 fluorophore in 5′).

Seven different conditions were tested:
initiator sequence+enzyme, no rNTP;
initiator sequence+enzyme+ATP;
initiator sequence+enzyme+AGP;
initiator sequence+enzyme+UTP;
initiator sequence+enzyme+CTP;
initiator sequence+enzyme, no dNTP;
initiator sequence+enzyme+dUTP;

PolpP12_{Δ297-898} was found to incorporate both ribonucleosides and deoxyuridine at 80° C., as demonstrated by the higher migration pattern of the initiator primer, when compared to the negative controls (FIG. 12).

This observation is all the more surprising that Gill et al., 2014 (Nucleic Acids Res. 42(6):3707-3719) showed that the DNA primase from Thermococcus nautili sp. 30-1 (having the amino acid sequence of SEQ ID NO: 10) and its truncated version PolpTN2_{Δ311-923} (having the amino acid sequence of SEQ ID NO: 11) both failed to incorporate ribonucleosides in a DNA primase activity assay.

### Example 6

PolpP12_{Δ297-898} is Capable of Incorporating Protected Ribonucleosides Triphosphate

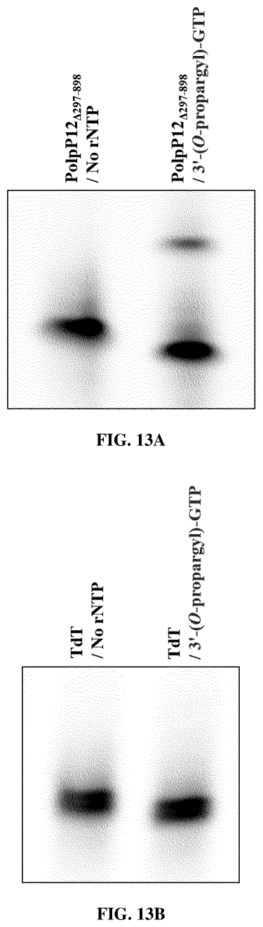

To further investigate the ability of PolpP12_{Δ297-898} to incorporate 3′-reversible terminating ribonucleotides, a terminal transferase activity assay was carried out at 70° C. using 3′-O-propargyl GTP (FIG. 13A) and a single stranded nucleic acid primer as initiator sequence (bearing a Cy5 fluorophore in 5′). For the sake of comparison, a similar experiment was carried out using the recombinant terminal deoxynucleotidyl transferase (TdT) from calf thymus at 37° C. (FIG. 13B).

For both experiments, two different conditions were tested:
initiator sequence+no enzyme+3′-O-propargyl GTP at 37° C.;
initiator sequence+calf thymus TdT+3′-O-propargyl GTP at 37° C.;
initiator sequence+no enzyme+3′-O-propargyl GTP at 70° C.;
initiator sequence+PolpP12_{Δ297-898+3}′-O-propargyl GTP at 70° C.

Interestingly, in contrast to calf thymus TdT, PolpP12_{Δ297-898} was found to incorporate 3′-O terminating ribonucleotides at 70° C., as demonstrated by the higher migration pattern of the initiator primer, when compared to negative controls (FIGS. 13A and B).

Thus, such an incorporation suggests that PolpP12_{Δ297-898} can be used for de novo RNA synthesis and further reinforces its usefulness for the industrial synthesis of nucleic acids.

### Example 7

PolpP12_{Δ297-898} is Capable of Incorporating Deoxyinosine Triphosphate and Base-Modified Nucleosides Triphosphate

Although the incorporation of sugar-modified nucleotides represents a major industrial concern, the synthesis of nucleic acids containing base analogs remains a critical feature for numerous biological applications. Indeed, some nucleotides such as inosine are usually employed for random mutagenesis experiments while biotin-modified bases serve as purification tag.

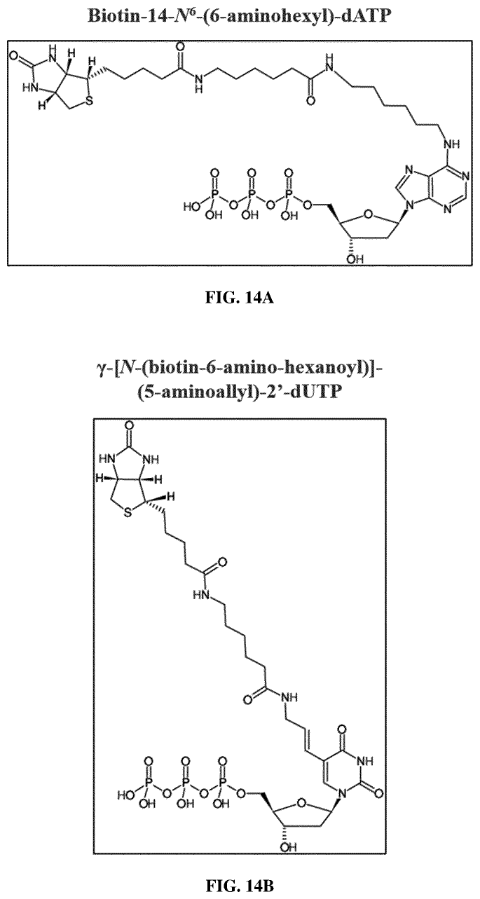

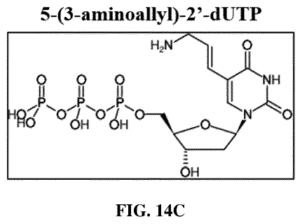

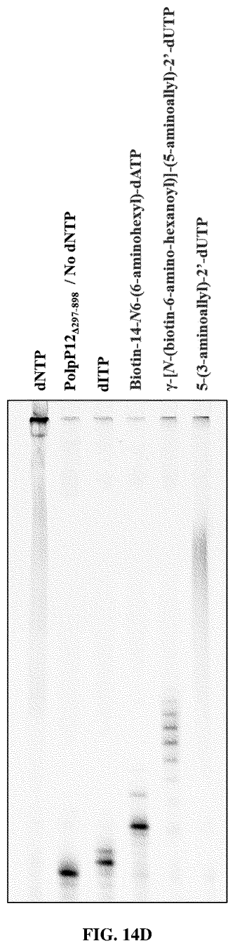

A terminal transferase activity assay was carried out with PolpP12_{Δ297-898} at 70° C. (FIG. 14) using deoxyinosine, biotin-14-N⁶-(6-aminohexyl)-dATP (FIG. 14A), γ-[N-(biotin-6-amino-hexanoyl)]-(5-aminoallyl)-2′-dUTP (FIG. 14B) or 5-(3-aminoallyl)-2′-dUTP (FIG. 14C), and a single stranded nucleic acid primer as initiator sequence (bearing a Cy5 fluorophore in 5′).

Six different conditions were tested:
initiator sequence+enzyme+dNTP at 70° C.;
initiator sequence only+enzyme, no dNTP at 70° C.;
initiator sequence+enzyme+deoxyinosine at 70° C.;
initiator sequence+enzyme+biotin-14-N⁶-(6-aminohexyl)-dATP at 70° C.;
initiator sequence+enzyme+γ-[N-(biotin-6-amino-hexanoyl)]-(5-aminoallyl)-2′-dUTP at 70° C.;
initiator sequence+enzyme+5-(3-aminoallyl)-2′-dUTP at 70° C.

PolpP12_{Δ297-898} was found to naturally incorporate all the tested base analogs nucleotides at 70° C., as demonstrated by the higher migration pattern of the initiator primer, when compared to the negative control without dNTP (FIG. 14D).

Incorporation of base-modified nucleotides represents another major industrial benefit as it allows a direct synthesis of chemically modified nucleic acids, which in turn saves time and efforts by limiting downstream modifications. For example, direct and controlled incorporation of one or multiple deoxyinosines in DNA coding sequences can be used to generate diversity during random mutagenesis and directed evolution experiments since this nucleotide is able to form wobble base pairs with adenosine, cytosine and uridine. Likewise, biotin is commonly used in molecular biology for both purification and detection. Indeed, biotin-modified nucleic acids can be purified and immobilized using streptavidin-agarose resins or detected and quantified using peroxidase-conjugated streptavidin. Nevertheless, when downstream modifications are still needed, direct incorporation of the aminoallyl group during de novo nucleic acids synthesis is of great importance. Indeed, this chemical modification facilitates specific nucleic acids labeling with biotin and dyes, using amino-reactive compounds, such as N-hydroxysuccinimide ester derivatives.

### Example 8

PolpP12_{Δ297-898} Variant with Internal Deletion is Still Functional

Although PolpP12_{Δ297-898}, PolpTN2_{Δ311-923}, PolpTCIR10_{Δ303-928}, PolpTpep_{Δ295-914} and PolpTcele_{Δ295-913} present similar activities, it is worth noting that these enzymes are diverging both in term of sequence identity and length. Indeed, protein sequence alignment of these enzymes showed the presence of a loop that we suspected might be dispensable for terminal nucleotidyl transferase activity in PolpP12_{Δ297-898}. This loop is located between amino acid residues 87 to 92 of PolpP12_{Δ297-898} (reference to SEQ ID NO: 2 numbering).

This study was driven by the necessity of providing an enzyme that is suitable for industrial applications, and adapted for both upstream and downstream processes. In that respect, the removal of this loop can improve on the one hand protein stability and protein expression yield as it maximizes the presence of structured regions. On the other hand, loop deletion leads to a reduced protein size, which eventually facilitates the removal of the enzyme along with other reagents by ultrafiltration during downstream purification.

To investigate the effect of loop deletion and size reduction on terminal nucleotidyl transferase activity, we generated variants of PolpTN2_{Δ311-923} (which itself also comprises not one but three loops located between amino acid residues 90 to 96, 205 to 211 and 248 to 254 of PolpTN2_{Δ311-923}, reference to SEQ ID NO: 11 numbering). In particular, the first loop located between amino acid residues 90 to 96 of SEQ ID NO: 11 corresponds to the loop located between amino acid residues 87 to 92 of SEQ ID NO: 2.

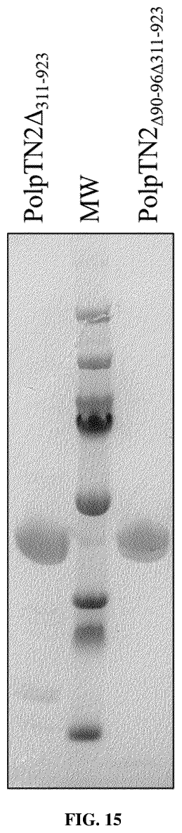

We thus produced among others a PolpTN2_{Δ90-96Δ311-923} variant lacking these amino acid residues 90 to 96, which was expressed and purified as previously described (FIG. 15), and we subsequently investigated the ability of this variant to perform a template-independent DNA synthesis reaction in the presence or in the absence of the initiator sequence (bearing the Cy5 fluorophore in 5′).

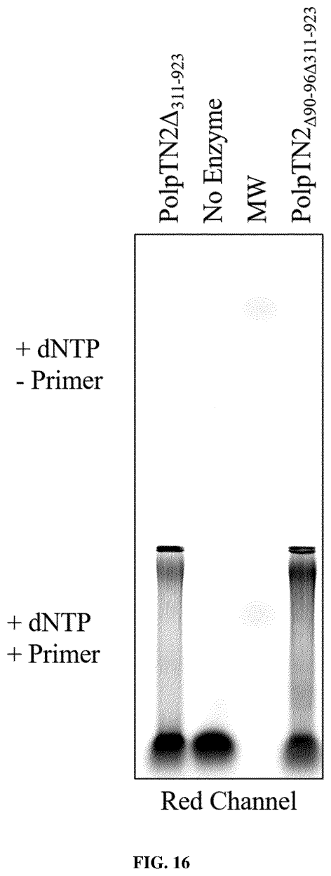

For that purpose, PolpTN2_{Δ90-96Δ311-923} and PolpTN2_{Δ311-923} (as control) were incubated at 70° C. with or without the initiator sequence and their terminal transferase activity was evaluated by following the polymerization of the fluorescent primer, recorded at 675 nm (red channel) (FIG. 16). Their ab-initio single-stranded nucleic acid synthesis activity was also evaluated through MidoriGreen direct staining and recorded at 520 nm (data not shown).

As seen on FIG. 16, PolpTN2_{Δ90-96Δ311-923} demonstrated an ability to extend single-stranded DNA fragments, similarly to PolpTN2_{ΔΔ311-923}. It also demonstrated an ab-initio activity (data not shown).

These results hence demonstrate the possibility of shaping these enzymes to optimally integrate them into industrial processes that require downstream steps, such as ultrafiltration.

Hence, since this loop deletion is not detrimental to the activity of the enzyme, it is expectable that the deletion of the corresponding loop in PolpP12_{Δ297-898} would also lead to a functional enzyme (PolpP12_{Δ87-92Δ297-898} with SEQ ID NO: 3).

### CONCLUSION

In conclusion, we were able to show that PolpP12_{Δ297-898} exhibit a template-independent nucleic acid synthesis activity in presence of an initiator primer and nucleosides triphosphate, whether unprotected or 3′-O protected, and regardless of the size of the protecting group. Interestingly, this template-independent nucleic acid synthesis activity was not only observed with deoxyribonucleotides but also with ribonucleotides, as well as with base-modified nucleosides triphosphate.

PolpP12_{Δ297-898} is a thermostable enzyme, which makes it especially useful for the synthesis of GC-rich sequences which necessitate temperatures higher than 60° C. to break their stable secondary structures during synthesis. It is also highly processive compared to the classically used TdT, since it was able to synthetize long strands of 1.5 kb at 70° C. when TdT could synthetize only small strands of 400 b at 37° C.

Moreover, PolpP12_{Δ297-898} does not exhibit an ab-initio nucleic acid synthesis activity, contrary to PolpTN2_{Δ311-923}, which competing activity may be detrimental in several nucleic acid synthesis processes.

Finally, PolpP12A87-92_{Δ297-898} is expected to display the same activity as PolpP12_{Δ297-898}, while being more stable and producible in higher amounts.

All these surprising properties and capabilities of PolpP12_{Δ297-898} make it thus an excellent resource for nucleic acid synthesis processes.

1. A method for template-independent synthesis of nucleic acids, comprising iteratively contacting an initiator sequence comprising a 3′-end nucleotide with a free 3′-hydroxyl group, with at least one nucleoside triphosphate, or a combination of nucleoside triphosphates, in the presence of a primase domain of the Pyrococcus sp. 12-1 DNA primase, or a functionally active fragment and/or variant thereof, thereby covalently binding said nucleoside triphosphate to the free 3′-hydroxyl group of the 3′-end nucleotide,
wherein said primase domain has an amino acid sequence of SEQ ID NO: 2 or SEQ ID NO:3, and
wherein said primase domain has an amino acid sequence of:
has at least 70% sequence identity with the amino acid sequence of SEQ ID NO: 2 or SEQ ID NO:3; and
is capable of template-independent terminal nucleotidyl transferase activity; and
is devoid of an ab-initio single-stranded nucleic acid synthesis activity.

2. The method according to claim 1, wherein said archaeal DNA primase or the functionally active variant thereof is from an archaeon of the Pyrococcus genus.

3. The method according to claim 1 or 2, wherein said archaeal DNA primase or the functionally active variant thereof is Pyrococcus sp. 12-1 DNA primase.

4. The method according to any one of claims 1 to 3, wherein said archaeal DNA primase belonging to the primase-polymerase family or the functionally active variant thereof is Pyrococcus sp. 12-1 DNA primase having the amino acid sequence of SEQ ID NO: 1.

5. The method according to claim 1, wherein said primase domain or functionally active fragment and/or variant thereof has the amino acid sequence of SEQ ID NO: 2, and wherein functionally active fragment and/or variant thereof:
has at least 70% sequence identity with the amino acid sequence of SEQ ID NO: 2; and
is capable of template-independent terminal nucleotidyl transferase activity; and
is devoid of an ab-initio single-stranded nucleic acid synthesis activity.

6. The method according to any one of claims 1 to 5, wherein said primase domain of an archaeal DNA primase belonging to the primase-polymerase family is the primase domain of the Pyrococcus sp. 12-1 DNA primase having the amino acid sequence of SEQ ID NO: 2, or a functionally active fragment and/or variant thereof:
having at least 70% sequence identity with the amino acid sequence of SEQ ID NO: 2; and
being capable of template-independent terminal nucleotidyl transferase activity; and
being devoid of an ab-initio single-stranded nucleic acid synthesis activity.

7. The method according to claim 1, wherein the initiator sequence is immobilized onto a support.

8. The method according to claim 1, wherein the initiator sequence is a single stranded nucleic acid primer.

9. The method according to claim 1, wherein the template-independent synthesis of nucleic acids is carried out at a temperature ranging from about 60° C. to about 95° C.

10. The method according to claim 1, wherein said method is for template-independent synthesis of nucleic acids with random nucleotide sequence, and the at least one nucleoside triphosphate, or the combination of nucleoside triphosphates, does not comprise terminating nucleoside triphosphates.

11. The method according to claim 1, wherein said method is for template-independent sequence-controlled synthesis of nucleic acids, and the at least one nucleoside triphosphate is a terminating nucleoside triphosphate comprising a reversible 3′-blocking group.

12. The method according to claim 11, comprising the steps of:
a) providing the initiator sequence comprising a 3′-end nucleotide with a free 3′-hydroxyl group;
b) contacting said 3′-end nucleotide with a reversibly terminating nucleoside triphosphate in the presence of the primase domain of the archaeal DNA primase belonging to the primase-polymerase family or the functionally active variant thereof,
thereby covalently binding said reversibly terminating nucleoside triphosphate to the free 3′-hydroxyl group of the 3′-end nucleotide;
c) applying a washing solution to remove all reagents, in particular to remove unbound reversibly terminating nucleoside triphosphates;
d) cleaving the reversible 3′-blocking group of the covalently bound terminating nucleoside triphosphate in the presence of a cleaving agent; and
thereby obtaining a nucleotide with a free 3′-hydroxyl group.

13. An isolated functionally active fragment of an archaeal DNA primase consisting of an amino acid sequence of SEQ ID NO: 2 or SEQ ID NO: 3, or a functionally active fragment and/or variant thereof:
having at least 70% sequence identity with the amino acid sequence of SEQ ID NO: 2 or SEQ ID NO: 3; and
being capable of template-independent terminal nucleotidyl transferase activity; and
being devoid of an ab-initio single-stranded nucleic acid synthesis activity.

14. The isolated functionally active fragment of the archaeal DNA primase or variant thereof according to claim 13, consisting of an amino acid sequence of SEQ ID NO: 2, or a functionally active fragment and/or variant thereof:
having at least 70% sequence identity with the amino acid sequence of SEQ ID NO: 2; and
being capable of template-independent terminal nucleotidyl transferase activity; and
being devoid of an ab-initio single-stranded nucleic acid synthesis activity.

15. The isolated functionally active fragment of the archaeal DNA primase or variant thereof according to claim 13, consisting of the amino acid sequence of SEQ ID NO: 2 or SEQ ID NO: 3.

16. A nucleic acid encoding the functionally active fragment of an archaeal DNA primase according to claim 13, or an expression vector comprising the nucleic acid operably linked to regulatory elements.

17. The nucleic acid according to claim 16, wherein the nucleic acid is operably linked to a promoter.

18. A host cell comprising the expression vector according to claim 17.

19. A method of producing the functionally active fragment of an archaeal DNA primase according to any one of claims 13 to 15, said method comprising:
(a) culturing the host cell according to claim 18, under conditions suitable for the expression of said functionally active fragment of the archaeal DNA primase or variant thereof; and
(b) isolating said functionally active fragment of the archaeal DNA primase or variant thereof from said host cell.

20. A kit comprising:
an initiator sequence comprising a 3′-end nucleotide with a free 3′-hydroxyl group, optionally immobilized onto a support;
at least one nucleoside triphosphate, optionally wherein the at least one nucleoside triphosphate is a terminating nucleoside triphosphate comprising a reversible 3′-blocking group; and
the isolated functionally active fragment of the archaeal DNA primase according to any one of claims 13 to 15.

21. The method according to claim 12, further comprising the step of:
(e) applying a washing solution to remove all reagents, in particular to remove the cleaving agent.

22. The method according to claim 21, further comprising the step of:
(f) reiterating steps b) to e) multiple times to synthetize the nucleic acid until desired length and nucleotide sequence.
